(19) United States
(12) Patent Application Publication
(10) Pub. No.: US 2026/0218931 A1
(43) Pub. Date: Jul. 30, 2026

(54) DROWSINESS LEVEL ESTIMATION DEVICE, APPARATUS INCLUDING DROWSINESS LEVEL ESTIMATION DEVICE, AND AIR-CONDITIONING APPARATUS

(71) Applicants: Mitsubishi Electric Corporation, Tokyo (JP); CAREA CORPORATION CO., LTD., Toyama City, Toyama (JP)

(72) Inventors: Reiji MORIOKA, Tokyo (JP); Kazuo YOSHIDA, Toyama (JP)

(21) Appl. No.: 19/144,767

(22) PCT Filed: Jan. 30, 2023
(86) PCT No.: PCT/JP2023/002795

§ 371 Date: Jun. 30, 2025

(51) Int. Cl. F24F 11/63; A61B 5/00; A61B 5/024; F24F 120/14

(52) U.S. Cl. CPC F24F 11/63; A61B 5/024; A61B 5/4809; A61B 5/742; F24F 2120/14

(57) ABSTRACT

A drowsiness level estimation device includes a Doppler sensor configured to detect a pulse wave of a user, and an analysis unit configured to analyze the pulse wave detected by the Doppler sensor. The analysis unit is configured to generate an index value by digitizing the pulse wave based on chaos analysis in which a source of analysis is a pulse waveform shift that is a time-series shift of a waveform of the pulse wave, estimate a drowsiness level of the user based on the index value, and output drowsiness level information related to the estimated drowsiness level.

### CROSS-REFERENCE TO RELATED APPLICATION

This application is a U.S. national stage application of PCT/JP2023/002795 filed Jan. 30, 2023, the contents of which are incorporated herein by reference.

### TECHNICAL FIELD

The present disclosure relates to a drowsiness level estimation device that estimates a drowsiness level that is the level of drowsiness of a user, an apparatus including the drowsiness level estimation device, and an air-conditioning apparatus.

### BACKGROUND

There is a drowsiness level estimation device that estimates the drowsiness level of a user based on indoor temperature information acquired by a temperature detection unit, an action that shows drowsiness and is determined based on a user image acquired by an imaging unit, the surface temperature of the user, and a database associated with age or sex (see, for example, Patent Literature 1). This drowsiness level estimation device estimates the drowsiness level under the assumption that the user rests the chin on the hand or takes other actions when the drowsiness level increases.

### PATENT LITERATURE

Patent Literature 1: Japanese Unexamined Patent Application Publication No. 2019-082282

The drowsiness level estimation device of Patent Literature 1 estimates the drowsiness level under such pre-assumption that the drowsiness level increases when the user rests the chin on the hand. However, the drowsiness level does not always increase when the user rests the chin on the hand. The drowsiness level estimation device of Patent Literature 1 has plenty of room for improvement to increase the estimation accuracy.

### SUMMARY

To solve the above problem, an object is to provide a drowsiness level estimation device that can estimate a drowsiness level with high accuracy, an apparatus including the drowsiness level estimation device, and an air-conditioning apparatus.

A drowsiness level estimation device according to an embodiment of the present disclosure includes a Doppler sensor configured to detect a pulse wave of a user, and an analysis unit configured to analyze the pulse wave detected by the Doppler sensor. The analysis unit is configured to generate an index value by digitizing the pulse wave based on chaos analysis in which a source of analysis is a pulse waveform shift that is a time-series shift of a waveform of the pulse wave, estimate a drowsiness level of the user based on the index value, and output drowsiness level information related to the estimated drowsiness level.

An apparatus according to another embodiment of the present disclosure includes the drowsiness level estimation device described above, and a controller configured to control an operation of an apparatus body based on the drowsiness level information output from the drowsiness level estimation device.

An air-conditioning apparatus according to still another embodiment of the present disclosure includes the drowsiness level estimation device described above, an air-conditioning unit configured to condition air in an indoor space, and a controller configured to control the air-conditioning unit based on the drowsiness level shown by the drowsiness level information output from the drowsiness level estimation device.

According to the embodiments of the present disclosure, the drowsiness level estimation device, the apparatus including the drowsiness level estimation device, and the air-conditioning apparatus can estimate the drowsiness level based on the index value generated based on the time-series shift of the waveform of the pulse wave detected by the Doppler sensor. The pulse wave is vital data related to heart pulsation and furthermore activities in a brain nervous system. The drowsiness level estimation device estimates the drowsiness level based on the index value obtained by digitization based on the pulse wave. Therefore, the drowsiness level can be estimated with high accuracy.

### BRIEF DESCRIPTION OF DRAWINGS

FIG. 1 is a block diagram illustrating the configuration of a drowsiness level estimation device according to Embodiment 1 and the use configuration of the drowsiness level estimation device.

FIG. 2 is a schematic diagram of an antenna surface of a Doppler sensor according to Embodiment 1.

FIG. 3 is a schematic diagram of a board component mounting surface of the Doppler sensor according to Embodiment 1.

FIG. 4 is a diagram illustrating an example of a pulse wave detected by the Doppler sensor according to Embodiment 1.

FIG. 5 is a diagram illustrating time-series data of a pulse waveform shift in the drowsiness level estimation device according to Embodiment 1.

FIG. 6 is a conceptual diagram of an attractor in chaos analysis in the drowsiness level estimation device according to Embodiment 1.

FIG. 7 is a conceptual diagram of Lyapunov exponentiation in the drowsiness level estimation device according to Embodiment 1.

FIG. 8 is a diagram illustrating, in the form of a bar graph, the relationship between Lyapunov exponents and various behaviors different in brain activities in a central nervous system.

FIG. 9 is a diagram illustrating an example of changes in the Lyapunov exponent during work.

FIG. 10 is a flowchart of a drowsiness level estimation process in the drowsiness level estimation device according to Embodiment 1.

FIG. 11 is an explanatory diagram of a WORF method used in known examples.

FIG. 12 is a block diagram illustrating the configuration of a drowsiness level estimation device according to Embodiment 2 and the use configuration of the drowsiness level estimation device.

FIG. 13 is a block diagram of an analysis unit of a drowsiness level estimation device according to Embodiment 3.

FIG. 14 is a conceptual diagram of a first-order short section drowsiness level, a second-order short section drowsiness level, and a long section drowsiness level calculated by the drowsiness level estimation device 1 according to Embodiment 3.

FIG. 15 is a flowchart illustrating an outline of a process in the analysis unit of the drowsiness level estimation device according to Embodiment 3.

FIG. 16 is a diagram illustrating the configuration of an air-conditioning apparatus according to Embodiment 4.

FIG. 17 is a block diagram of the air-conditioning apparatus according to Embodiment 4.

FIG. 18 is a flowchart illustrating an operation of the air-conditioning apparatus according to Embodiment 4.

### DETAILED DESCRIPTION

Embodiments 1 to 4 of the present disclosure are described below with reference to the drawings. In the drawings, the same or corresponding parts are denoted by the same reference signs and their description is omitted or simplified as appropriate.

### Embodiment 1

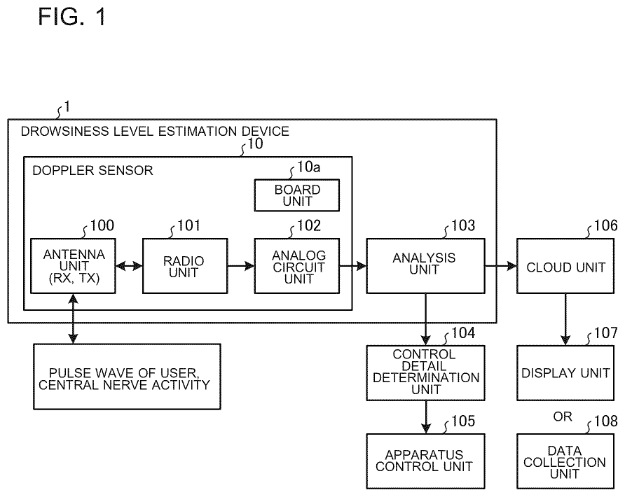

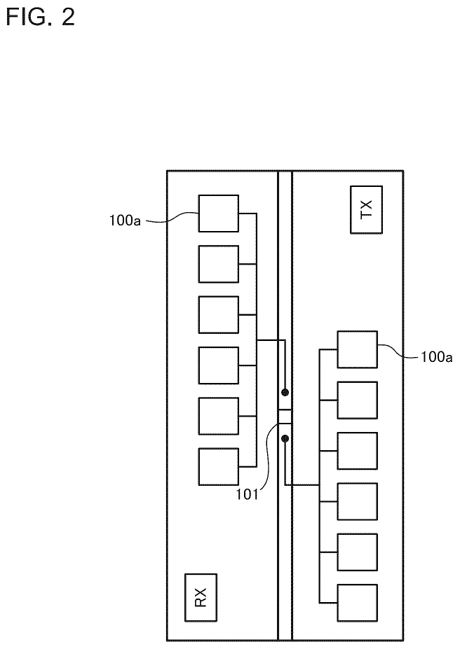

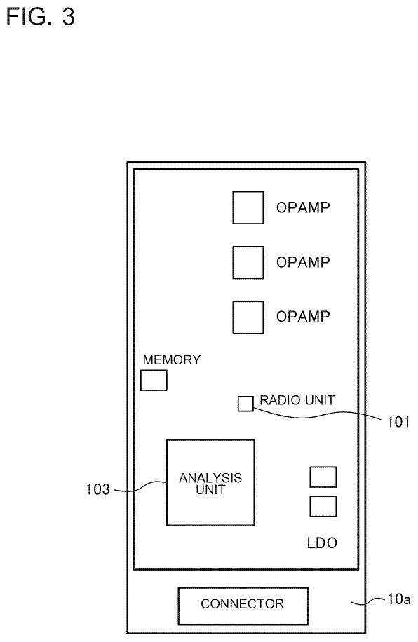

FIG. 1 is a block diagram illustrating the configuration of a drowsiness level estimation device 1 according to Embodiment 1 and the use configuration of the drowsiness level estimation device 1. FIG. 2 is a schematic diagram of an antenna surface of a Doppler sensor 10 according to Embodiment 1. FIG. 3 is a schematic diagram of a board component mounting surface of the Doppler sensor 10 according to Embodiment 1. The drowsiness level estimation device 1 estimates the drowsiness level of a user. The drowsiness level estimation device 1 shows the drowsiness level in the numerical form to objectify the drowsiness level.

The drowsiness level estimation device 1 includes the Doppler sensor 10 and an analysis unit 103. The Doppler sensor 10 emits a constant sinusoidal radio wave at about 24 GHz called “microwave band” or “submillimeter-wave band” toward a user whose drowsiness level is to be estimated. The body surface of the user is displaced by movement of blood vessels due to changes in a bloodstream along with heart pulsation. When the distance between the body surface of the user and the Doppler sensor 10 changes, the reflection wave reflected by the body surface of the user changes due to the Doppler effect.

The Doppler sensor 10 receives the reflection wave from the user in response to the movement of the blood vessels, and detects a pulse wave in the central nervous system of the user based on a frequency difference between the reflection wave and the transmission wave emitted from the Doppler sensor 10. The pulse wave is a waveform showing a change in movement of the body surface of a person due to heart pulsation, and includes a waveform of a change in movement of blood vessels and a waveform of a change in the body surface at the heart portion. The blood vessels run all over the body of the user, and the Doppler sensor 10 can detect the movement of the blood vessels in part of the body of the user, such as part of the head or arm, instead of the heart.

In distance measurement, a measurement frequency of 60 to 79 GHz is often used because of high resolution. However, the purpose in this case is detection of a pulse wave unlike great body movement or other movement, and it is necessary to analyze an infinitesimal fluctuation having an extremely low frequency characteristic of about 1 Hz. Therefore, analog detection using the 24-GHz Doppler system is suitable for the pulse wave detection.

The Doppler sensor 10 is advantageous in that the pulse wave of the user can be detected using the above radio wave without contact. Thus, the Doppler sensor 10 can measure a wide range of the body surface of the user. The Doppler sensor 10 can measure vital data such as a pulse rate, a respiration rate, body movement, a sleep state, and an autonomic balance by analyzing peak intervals of the pulse wave, but is used to detect the pulse wave in Embodiment 1.

Pulse wave sensors include not only the Doppler sensor that is a non-contact sensor, but also a contact sensor that performs detection while being in contact with the user. There are many devices that measure pulses by contact. A photoelectric pulse wave sensor is often used as a wearable device. The pulse wave sensor detects a change in the volume of a blood vessel along with blood pumping from the heart as a waveform, and includes a detector that monitors the volume change. The pulse wave sensor can obtain a pulse interval by measuring an interval between the peaks of the obtained pulse wave. A pulse rate per minute can be calculated by determining an inverse of the pulse interval. For example, when the pulse interval is 800 ms (0.8 seconds) on average, the pulse rate is 75 per minute based on 60÷0.8.

The pulse wave sensors include a transmission type and a reflection type depending on a difference in measurement method. The transmission pulse wave sensor can measure a pulse wave by irradiating the body surface with an infrared ray or red light and measuring a change amount of light passing through the body as a change in the blood flow rate along with heart pulsation. However, the measurable portion for the transmission pulse wave sensor is limited to a portion through which the infrared ray or red light easily passes, such as a fingertip or an ear lobe.

The reflection pulse wave sensor irradiates a living body with an infrared ray, red light, or light having a green wavelength of about 550 nanometers, and measures light reflected in the living body using a photodiode or phototransistor. Oxyhemoglobin is present in artery blood, and has a characteristic that it absorbs incident light. Therefore, the reflection pulse wave sensor can measure a pulse wave signal by sensing, in time series, a blood flow rate that changes along with heart pulsation (change in pressure in the blood vessel). Specifically, the pulse wave sensor includes a light emitting element and a light receiving element. The light emitting element radiates light, and the light receiving element detects light reflected by a finger. The intensity of the light reflected by the finger shows an increase or decrease amount of hemoglobin flowing through a capillary vessel of the fingertip. The reflection pulse wave sensor can obtain time-series pulse wave data associated with the increase or decrease amount of hemoglobin (blood flow rate). Since the reflection pulse wave sensor performs measurement using the reflected light, there is no need to limit the measurement portion like the transmission pulse wave sensor.

However, the contact sensor such as the pulse wave sensor needs to perform measurement while being worn. Therefore, there are problems in that the wearing is bothersome and measurement cannot be performed remotely. In view of this, the drowsiness level estimation device 1 uses the non-contact Doppler sensor 10 as the sensor that measures the pulse wave of the user. With the Doppler sensor 10, the drowsiness level estimation device 1 can acquire the pulse wave of the user without contact, and the user need not wear the sensor. Thus, data necessary for drowsiness level estimation can be acquired without bothering the user with the wearing of the sensor. With the Doppler sensor 10, the drowsiness level estimation device 1 can measure the pulse wave in a wide range of the body surface of the user.

The Doppler sensor 10 mixes an emitted signal with a received signal, extracts a variation component caused by the Doppler effect, and generates a pulse wave of an IQ signal. Specifically, the Doppler sensor 10 includes an antenna unit 100, a radio unit 101, an analog circuit unit 102, and a board unit 10a. The antenna unit 100 acquires a pulse wave of the user that is a central nerve activity. The antenna unit 100 includes an emission unit TX and a reception unit RX. As illustrated in FIG. 2, the antenna unit 100 includes a plurality of (12 in this case) antennas 100a. TX and RX each include six antennas 100a.

The radio unit 101 generates a radio wave at 24 GHz called “radio frequency (RF),” emits the radio wave from TX, and receives the reflection wave by RX. The analog circuit unit 102 includes a circuit unit that performs IQ detection on the reflection wave, converts the wave into an IQ signal, and converts a frequency component that is a Doppler change in the reflection wave. As illustrated in FIG. 3, the analog circuit unit 102 includes an analog amplification filter unit (OPAMP) that extracts and amplifies a necessary frequency band, and an analog-digital conversion unit (LDO) that enables numerical analysis.

The board unit 10a includes a connector unit for outputting information to the analysis unit 103 or an apparatus including the drowsiness level estimation device 1, and a memory. As illustrated in FIG. 3, the radio unit 101, the analog circuit unit 102, and the analysis unit 103 are covered with a metal shield case. In FIG. 3, the shield case part is dotted.

The analysis unit 103 analyzes the pulse wave detected by the Doppler sensor 10. The analysis unit 103 generates an index value by digitizing the pulse wave based on chaos analysis in which the source of analysis is a pulse waveform shift that is a time-series shift of the waveform of the pulse wave (hereinafter referred to as “pulse waveform”), and estimates the drowsiness level based on the index value. The drowsiness level estimation by the analysis unit 103 is described later. The analysis unit 103 outputs drowsiness level information showing a drowsiness level estimation result. The drowsiness level information is information related to the drowsiness level. The drowsiness level information output from the analysis unit 103 is input to a control detail determination unit 104 or a cloud unit 106 described later.

The analysis unit 103 is a microprocessor unit. The analysis unit 103 includes a central processing unit (CPU), a read only memory (ROM), and a random access memory (RAM). The ROM stores a control program or other data. The analysis unit 103 is not limited to the microprocessor unit. For example, the analysis unit 103 may be firmware that can be updated. The analysis unit 103 may be a program module to be executed by a command from a CPU (not illustrated) or other devices. The analysis unit 103 may separately be provided outside the Doppler sensor 10, or may be provided on the board in the Doppler sensor 10 to perform edge processing in the single sensor.

The drowsiness level estimated by the analysis unit 103 can be used to control an apparatus including the drowsiness level estimation device 1. The apparatus including the drowsiness level estimation device 1 is, for example, an air-conditioning apparatus, and details thereof are described later in Embodiment 4. The apparatus including the drowsiness level estimation device 1 includes the control detail determination unit 104 and an apparatus control unit 105. The control detail determination unit 104 determines details of control on the apparatus based on the input drowsiness level information, generates control data, and outputs it to the apparatus control unit 105. The apparatus control unit 105 controls various actuators of the apparatus based on the control data from the control detail determination unit 104.

As a use form of the drowsiness level estimation device 1, the drowsiness level information output from the analysis unit 103 may be input to the cloud unit 106. The cloud unit 106 accumulates the drowsiness level information input from the analysis unit 103. The analysis unit 103 may further output vital data that is pulse wave data obtained by the Doppler sensor 10, and the cloud unit 106 may accumulate it. The drowsiness level information and the vital data accumulated in the cloud unit 106 can be visualized when displayed on a display unit 107. The drowsiness level information and the vital data accumulated in the cloud unit 106 need not always be visualized, and may be provided to a data collection unit 108 from another cloud via the cloud unit 106 and utilized for various purposes.

The display unit 107 is a display such as a liquid crystal display panel. The display unit 107 may be a display unit of a smartphone, and the drowsiness level information may be visualized by being displayed on an application installed in the smartphone.

In the conventional system that estimates the drowsiness level of a person from an image acquired by the imaging unit, the drowsiness level is estimated under such pre-assumption that the drowsiness level increases when the person rests the chin on the hand. However, the drowsiness level does not always increase when the person rests the chin on the hand. This system has room for improvement to increase the estimation accuracy. Hitherto, there is a device that estimates human emotions using an electroencephalograph, but a waves and p waves of brain waves are not easily quantified. The estimation device using the electroencephalograph has such problems in terms of practicality and analysis time that the estimation cannot be performed in real time because measurement data in a predetermined period is required for the estimation process, the electroencephalograph needs to be worn on the head, and the system is complicated.

The drowsiness level estimation device 1 of Embodiment 1 can estimate the drowsiness level in a short time by the chaos analysis described below from the pulse wave detected by the Doppler sensor 10 without contact.

Details of the chaos analysis are described. In physiopsychology, the physiological state and the psychological state of a person are estimated from a physiological change exhibited in a biometric signal. In the conventional physiopsychology, various biometric signals such as brain waves, an electrocardiogram, heartbeat intervals, blood pressure, and a respiration digital plethysmogram have been analyzed by various methods and many findings have been obtained. However, a majority of the analyses was mainly performed by analysis methods based on linear theory. However, the biometric signals include nonlinear characteristics and are known to vary due to nonlinear characteristics called “chaos.” Chaos refers to a phenomenon that the state transition rule of a system is deterministic but the nonlinearity of the system causes complexity equivalent to a stochastic system. The state of a target is deterministically described by, for example, an equation, but no rule is found in the phase of the state of the target and the phase shows a significantly complex behavior such as randomness.

Although the chaos phenomenon appears to have no order, this phenomenon definitely has a rule on the background in actuality. In other words, the phenomenon that will occur next is not determined by the probability but is deterministic in accordance with a predetermined rule. The reason why the target appears to have no order despite the rule is that the motions of elements of the target are simple but turn complex when they behave as an aggregate. The biometric signals generated from the complex system are determined to have a strong possibility that chaos information is present.

In recent years, various experiments have demonstrated effectiveness of chaos analysis in which the physiopsychological states of a person are estimated. In the conventional chaos analysis, information that is not determined uniquely, such as a thermal sensation or a psychological state of a person, is subjected to the chaos analysis as a chaos target, thereby finding a relationship from information that appears to have no relationship. In the chaos analysis, it is important to determine what is set as the chaos target. When the target changes, it leads to a different concept.

The chaos target of the drowsiness level estimation device 1 is a brain activity in the central nervous system. Hitherto, the brain activity in the central nervous system has not been the chaos target. The drowsiness level is deeply related to the brain activity in the central nervous system. Therefore, the drowsiness level estimation device 1 sets the brain activity in the central nervous system as the chaos target, and estimates the drowsiness level based on the result of the chaos analysis. The drowsiness level estimation device 1 performs the chaos analysis in which the source of analysis is a pulse waveform fluctuation focusing on how the waveform is shifted. The pulse waveform fluctuation is represented by time-series data of a shift of the waveform of the pulse wave, in other words, time-series shift data of the waveform of the pulse wave. The drowsiness level estimation device 1 generates an index value by digitizing the pulse wave based on the chaos analysis in which the source of analysis is a pulse waveform shift that is the time-series shift of the waveform of the pulse wave, and estimates the drowsiness level of the user based on the index value.

The chaos analysis has the following three sequential steps (1) to (3). Details of the steps are described later.
(1) The first step is a step of calculating vectors determined from the time-series data of the pulse waveform shift and a preset delay time.
(2) The second step is a step of generating an attractor having the vectors arranged in time series in a three-dimensional state space.
(3) The third step is a step of calculating a Lyapunov exponent that is the index value based on trajectories of the attractor.

The heart pulsation interval varies at every heartbeat. The variation originates from the brain and communication is made to the heart through the autonomic nerve. The inventors have considered that, in the drowsiness level estimation, the central nervous system as well as the autonomic nervous system is related to the heart pulsation, and conceived that the drowsiness level is finally estimated by finding a correlation between the heart pulsation and the brain activity level.

Hitherto, there is a technology of estimating a brain arousal level using a pulse change. The brain arousal level can be regarded as an index inverse to the drowsiness level. In the technology of estimating the brain arousal level, for example, determination is made that the arousal level is high when the pulse change is large, and that the arousal level is low and drowsiness occurs when the pulse change is small. The pulse change is a time variation in the interval between the peaks of the pulse wave, and is detected using only peak information of the pulse wave with pinpoint accuracy. The time variation in the interval between the peaks of the pulse wave may be hereinafter referred to as “one-dimensional pattern pulse shift” or “pulse fluctuation.” The term “one-dimensional” is used because the height of the pulse wave is not related to the time variation in the interval between the peaks of the pulse wave and vital data is obtained by frequency conversion using only the shift of the pulse interval.

Although the conventional technology of measuring the arousal level uses the pulse change, the inventors have considered that information on the complex nerve activity of the living body is not fully determined from the pulse change alone. The inventors have sought for a method different from the pinpoint detection using only the peak information of the pulse wave, and focused on the shift of the pulse waveform. Comparing the complexity of the peak interval fluctuation with the complexity of the two-dimensional waveform pattern fluctuation from waveform to waveform in the pulse wave, however, the two-dimensional waveform pattern fluctuation (i.e., pulse waveform fluctuation) is much more complex than the one-dimensional pulse interval fluctuation. This may be because the pulse waveform fluctuation is related to neuron activities of six cerebral cortices. Therefore, the inventors use, instead of the conventional analysis method, chaos as the method for analyzing the brain activity and furthermore the drowsiness level.

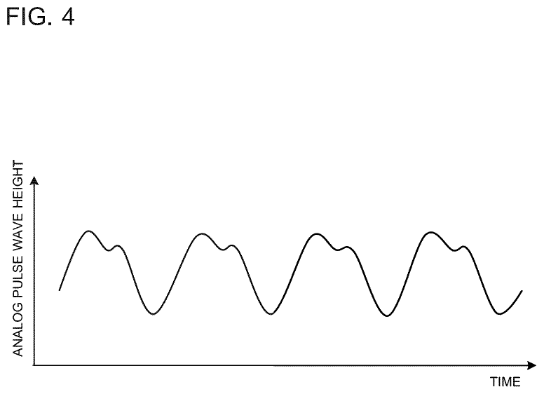

Next, the chaos analysis to be performed by the analysis unit 103 of the drowsiness level estimation device 1 is described. First, the analysis unit 103 acquires a pulse wave from the Doppler sensor 10. FIG. 4 illustrates an example of the pulse wave detected by the Doppler sensor 10.

FIG. 4 is a diagram illustrating an example of the pulse wave detected by the Doppler sensor 10 according to Embodiment 1. In FIG. 4, the horizontal axis represents time and the vertical axis represents an analog pulse wave height. The pulse wave height is power. The term “pulse wave height” does not refer to power at the peak where the power of the pulse wave is highest, but refers to time-based power in time series including the peak of the pulse wave. The Doppler sensor 10 obtains the analog waveform illustrated in FIG. 4 and outputs it to the analysis unit 103. The pulse wave height decreases as the distance between the Doppler sensor 10 and the measurement target user increases. In the chaos analysis of Embodiment 1, however, the shift of the pulse wave height is analyzed and therefore the absolute value of the pulse wave height is not necessary information in the pulse wave digitization. However, the pulse waveform is clearer when the pulse wave height increases, and the accuracy of attractor generation described later increases. Thus, the Doppler sensor 10 is preferably closer to the user.

The analysis unit 103 may perform the following to obtain a pulse wave height necessary to secure the accuracy in the analysis of the time-series data of the pulse waveform. The analysis unit 103 calculates a deviation of the pulse wave height. When the deviation is smaller than a preset threshold, the analysis unit 103 automatically adjusts the input signal amplification factor to virtually increase the size of the analog pulse waveform. In this manner, the shape of the pulse wave is clearer and the analysis unit 103 can perform highly accurate analysis even if the Doppler sensor 10 is located away from the user.

For example, when the Doppler sensor 10 is close to the user, the input signal multiplication factor may be “1.” As the distance between the Doppler sensor 10 and the user increases, the size of the pulse wave decreases, the shift of the pulse waveform is more unclear, and the deviation decreases. Therefore, the analysis unit 103 may determine the multiplication factor depending on how much the deviation is small. For example, when the deviation is smaller than a first threshold, the analysis unit 103 may set the input signal multiplication factor to “2,” and when the deviation is smaller than a second threshold that is smaller than the first threshold, the analysis unit 103 may set the input signal multiplication factor to “3.” That is, the analysis unit 103 increases the size of the analog waveform by automatically increasing the input signal amplification factor as the distance between the Doppler sensor 10 and the user increases. Thus, the analysis unit 103 can analyze the pulse waveform fluctuation (time-series shift data of the pulse waveform) even if the Doppler sensor 10 is located away from the user.

When the area of a child user or other users and the power of the bloodstream to blood vessels are small even at the same distance between the Doppler sensor 10 and the user, the analysis is difficult because the pulse wave level is low. Therefore, the analysis unit 103 increases the size of the analog waveform by automatically increasing the input signal amplification factor as the area of the user decreases and the deviation of the pulse wave height decreases. Thus, the analysis unit 103 can solve the problem that the analysis is difficult when the pulse wave level is low.

Next, the analysis unit 103 generates an attractor described later from the pulse waveform. The pulse waveform is a two-dimensional waveform pattern of the pulse wave. Although it is difficult to find a rule in the time-series data of the pulse waveform from waveform to waveform in the pulse wave, a predetermined pattern is present when the time-series data of the pulse waveform is converted into the attractor. The attractor is an aggregate in which a certain dynamical system temporally develops toward it. When motion is performed in the certain dynamical system from a point sufficiently close to the attractor, the system remains sufficiently close to the attractor. The trajectory included in the attractor has no limitation except that it remains in the attractor.

Next, the first to third steps of the chaos analysis to be performed by the analysis unit 103 are described in sequence. Embodiment 1 is characterized in that the brain activity in the central nervous system is the chaos target and the source of the chaos analysis is the pulse waveform shift that is the time-series shift of the waveform of the pulse wave. A conventionally known method is used as the method for the chaos analysis. Therefore, the chaos analysis is outlined below.

### (First Step)

As described above, the first step is the step of calculating vectors determined from the time-series data of the pulse waveform shift and the preset delay time.

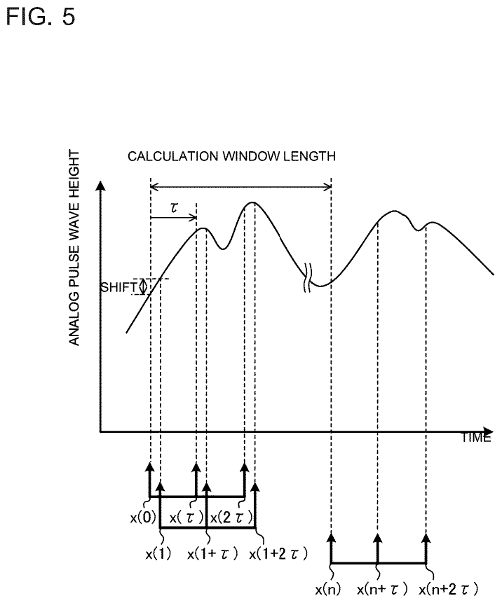

FIG. 5 is a diagram illustrating the time-series data of the pulse waveform shift in the drowsiness level estimation device 1 according to Embodiment 1. In FIG. 5, the horizontal axis represents time and the vertical axis represents the analog pulse wave height. x(i) (i=1, 2, . . . , n) is the time-series data of the pulse waveform based on sensor data obtained by the Doppler sensor 10. The difference between the pulse wave height at x(i) and the pulse wave height at x(i+1) is the pulse waveform shift. x(0) is an initial value of sensor data obtained within a time of a calculation window length. The calculation window length is the length of time set as appropriate, and is, for example, 20 seconds. The calculation can be performed more quickly as the calculation window length decreases. The amount of pulse waveform data increases and the accuracy increases as the calculation window length increases.

To embed the two-dimensional time-series change in a d-dimensional state space using the time-series data, in other words, to draw a locus in the d-dimensional state space, the analysis unit 103 generates vectors while setting a time lag T as an appropriate delay time. Specifically, the analysis unit 103 generates vectors X(i)={x(i), x(i+τ), x(i+2τ), . . . , x(i+(d−1)τ)}. For example, when d is “3” and the two-dimensional time-series change is embedded in a three-dimensional state space, the analysis unit 103 generates vectors with three state variables. In the three-dimensional state space, the vectors X(i) are X(i)={x(i), x(i+τ), x(i+2τ)}. Since i=1, 2, . . . , n, n vectors X are generated. The parameter T is called “embedding delay time.”

### (Second Step)

The second step is the step of generating an attractor having the vectors arranged in time series in a multidimensional state space such as a three-dimensional or higher-dimensional state space.

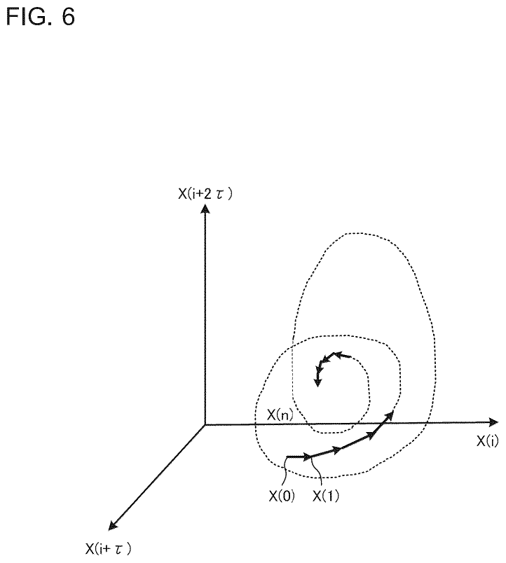

FIG. 6 is a conceptual diagram of the attractor in the chaos analysis in the drowsiness level estimation device 1 according to Embodiment 1. A trajectory is obtained when the vectors X(i) are sequentially plotted on coordinate axes x(i), x(i+τ), x(i+2τ), . . . , x(i+(d−1)τ). FIG. 6 illustrates a three-dimensional state space and three coordinate axes x(i), x(i+τ), and x(i+2τ). For example, when the vectors obtained in the first step are plotted (arranged) in time series in the three-dimensional state space with d=3 and the delay time set to 0.05 seconds, a locus as illustrated in FIG. 6 is obtained. This locus is the trajectory of the attractor. As for the shape of the trajectory of the attractor, a spiral locus is obtained. This demonstrates that chaos information is present in the fluctuation in the two-dimensional pattern pulse waveform.

### (Third Step)

The third step is the step of calculating a Lyapunov exponent that is an index value based on the trajectories of the attractor. The Lyapunov exponent is obtained by evaluating instability or divergence of the trajectories of the attractor.

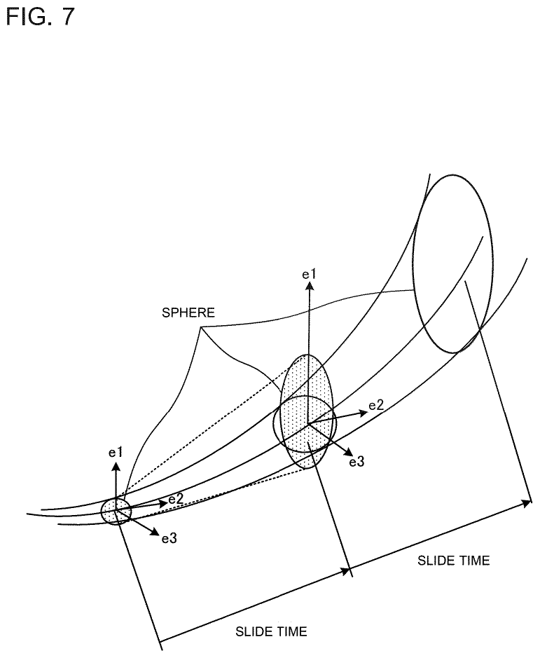

FIG. 7 is a conceptual diagram of Lyapunov exponentiation in the drowsiness level estimation device 1 according to Embodiment 1. FIG. 7 illustrates a case where the multidimensional state space is a three-dimensional state space. It is necessary to digitize the time-series shift of the pulse waveform to finally estimate the drowsiness level. The Lyapunov exponent is used as the index value obtained by the digitization. The attractor in the multidimensional state space such as a three-dimensional or higher-dimensional state space has instability of the trajectories. The instability can be rephrased as divergence. The Lyapunov exponent is obtained by quantifying the trajectory instability. The Lyapunov exponent is a measure of the distance between two trajectories departing from two nearby points. In other words, the Lyapunov exponent is a value showing the degree of separation of nearby trajectories in the dynamical system. As the Lyapunov exponent increases, the variation range of the attractor increases and the range of fluctuation increases.

In the calculation of the Lyapunov exponent, a microsphere (hypersphere) having a radius s is first given as an initial value to the trajectories of the attractor in a three-dimensional chaos dynamical system as illustrated in FIG. 7. Specifically, a sphere having the small radius s about a point on the trajectory of the attractor is set as the hypersphere on the trajectory. In the chaos analysis, points in the hypersphere are found on the trajectory of the attractor, and a change amount from each point to a point after an elapse of a slide time is linearly approximated. Thus, enlargement factors in e1, e2, and e3 directions are calculated. The slide time is a time interval from a previous sphere to a subsequent sphere. The hypersphere is initially a sphere. After an elapse of a preset slide time S, mapping is performed once so that the sphere is extended in the e1 direction, substantially unchanged in the e2 direction, and contracted in the e3 direction. As a result, the sphere becomes an ellipse. Assuming that λ1, λ2, and λ3 are logarithms of the enlargement factors per unit time in the e1, e2, and e3 directions, λ1, λ2, and λ3 are Lyapunov exponents.

The set of the Lyapunov exponents is called “Lyapunov spectrum.” In the chaos analysis, the operation of stretching the sphere is repeated at intervals of the slide time S and the enlargement factors are calculated. The sums are obtained and averaged so that an overall Lyapunov spectrum is calculated. The largest Lyapunov exponent among the calculated Lyapunov exponents is called “maximum Lyapunov exponent.” The maximum Lyapunov exponent shows the degree of separation of the trajectories compared with the initial state in the dynamical system. That is, the Lyapunov exponent shows the degree of separation in the n-th state compared with the initial state. As for the brain activity, the human brain works more actively as the degree of separation increases compared with the initial state, and the brain works inactively when the degree of separation remains unchanged compared with the initial state. Although the accuracy decreases, the Lyapunov exponents may be calculated by comparison between two states with the slide time regarded as one section instead of calculation performed multiple times at intervals of the slide time S.

When the slide time is reduced in the chaos analysis, the calculation accuracy is improved but the calculation time increases. When the number of points in the sphere is excessively large, the calculation time increases. Therefore, a nearby point count may be set as an upper limit value of the number of points in the sphere to reduce the calculation time. When the count of points in the sphere is larger than the set upper limit count, the calculation proceeds to the subsequent sphere.

Although the description has been made about the case where the multidimensional state space is the three-dimensional state space, the analysis unit 103 can generate the attractor in a three or higher-dimensional state space. When the multiple dimensions are five or more dimensions, the accuracy increases but the calculation time increases. Thus, practicality decreases. In view of the obtainment of chaoticness and the calculation time, the multiple dimensions are preferably three or four dimensions.

The description has been made that the Lyapunov exponent shows the degree of separation of the trajectories. The relationship between the degree of separation of the trajectories and the brain activity shows that, as the trajectories separate compared with the initial state, the human brain works actively and the brain activity level is high. When the trajectories remain unchanged compared with the initial state, the brain works inactively and the brain activity level is low.

Specifically, the Lyapunov exponent is calculated as follows. For example, it is assumed that the radius of the sphere is 0.08, the calculation window length is 20 seconds, the slide time is 1 second, and the measurement frequency is 200 Hz. Since the measurement frequency is 200 Hz, 200 pieces of data are obtained within 1 second, and 4000 pieces of data are obtained within the time of the calculation window length from the start of calculation. As for the Lyapunov exponent in the three-dimensional state space, a Lyapunov spectrum including λ1, λ2, and λ3 in three dimensions is obtained using 4000 pieces of data within 20 seconds from the start of measurement. A Lyapunov spectrum including λ1, λ2, and λ3 is similarly obtained within the next 1 second.

When an output window length is set to 60 seconds, the above operation is repeated for 60 seconds. That is, the first Lyapunov spectrum is obtained within the first 20 seconds, and a Lyapunov spectrum is obtained every 1 second of the slide time within the next 40 seconds. Thus, a total of 40 Lyapunov spectra are obtained. Then, the sums of λ1, λ2, and λ3 in the 40 Lyapunov spectra are obtained and averaged, and the averaged λ1, λ2, and λ3 are determined. The largest Lyapunov exponent among them is the maximum Lyapunov exponent. The maximum Lyapunov exponent is one piece of data obtained within the output window length. That is, the maximum Lyapunov exponent is employed as the degree of separation compared with the initial value in the system.

The number of measured pieces of pulse wave data that is vital data is discussed. For example, when the measurement frequency is set to 200 Hz and the output window length is set to 60 seconds, 12000 pieces of data are obtained within 60 seconds. The analysis unit 103 generates the trajectory of the attractor using the 12000 pieces of data, and calculates the Lyapunov exponent. As described above, the output window length is a time necessary to output one piece of data, and is a time necessary to calculate one Lyapunov exponent. For example, when the measurement frequency is set to 500 Hz and the output window length is set to 60 seconds, 30000 pieces of data are obtained within 60 seconds. In this case, the analysis unit 103 generates the trajectory of the attractor using the 30000 pieces of data, and calculates the Lyapunov exponent.

As the number of pieces of data increases, there is an advantage in that the accuracy increases. When the number of pieces of data is large, however, there are disadvantages in that the measurement time increases, the drowsiness level change cannot be followed, and the CPU processing time increases or calculation cannot be performed. As the number of pieces of data decreases, the accuracy decreases. When the number of pieces of data is small, however, there are advantages in that the measurement time decreases, the CPU processing time decreases, and the followability is good.

Thus, the measurement frequency is preferably about 100 Hz to 1000 Hz. The output window length is preferably 30 seconds or more and less than 5 minutes. Since the response time in the central nervous system is short, the output window length is preferably shorter than in the case of measurement in the autonomic nervous system, that is, preferably 30 seconds or more and 60 seconds or less. The analysis unit 103 may perform the following to increase the accuracy. The analysis unit 103 may collect one output piece of data in every set time and perform averaging. For example, when the output window length is 30 seconds and the set time is 5 seconds, the analysis unit 103 collects, at a timing of 5 seconds, one piece of data output based on vital data in 30 seconds before the timing. When 30 pieces of data are collected, the analysis unit 103 may obtain one piece of data by averaging the 30 pieces of data. In this case, the analysis unit 103 can obtain one piece of highly accurate data (Lyapunov exponent) taking 3 minutes for digitization.

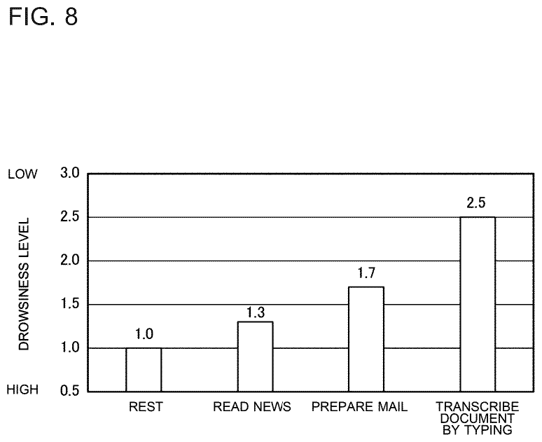

Next, the correlation between the “Lyapunov exponent” found by the inventors and the “brain activity in the central nervous system” deeply related to the drowsiness level is described with reference to FIG. 8. FIG. 8 is a diagram created based on results of experiment in which the user performs various behaviors different in brain activities and Lyapunov exponents are calculated based on pulse waves acquired from the user performing the behaviors.

FIG. 8 is a diagram illustrating, in the form of a bar graph, the relationship between the Lyapunov exponents and the various behaviors different in brain activities in the central nervous system. Numerical values on the vertical axis are values normalized with a rest set to “1.” That is, the numerical values on the vertical axis in the behaviors other than the rest are values obtained by dividing the Lyapunov exponents of the behaviors by the Lyapunov exponent of the rest. The horizontal axis represents human behaviors with different brain activity levels. The following four behaviors are employed as the behaviors on the horizontal axis. The four behaviors are “taking rest,” “reading news,” “preparing mail,” and “transcribing document by typing.” For example, “reading news” refers to a behavior of reading a document on a personal computer or a smartphone. “Transcribing document by typing” refers to a behavior of typing texts in a document on a personal computer.

FIG. 8 demonstrates that the numerical value on the vertical axis is larger in the heavy work such as “preparing mail” or “transcribing document by typing” in which the brain activity level is high even according to the user's subjective report than in the light work such as “taking rest” or “reading news.” That is, the Lyapunov exponent is larger when the brain activity level is higher. Thus, the brain activity level in the central nervous system and the Lyapunov exponent have a relationship.

During the rest, the drowsiness level is higher than during the transcription of a document by typing. This is also shown in the user's subjective report. Thus, the behavior, the Lyapunov exponent, and the drowsiness level have a correlation. The Lyapunov exponent may be handled also as a mental index that is the drowsiness level. Specifically, evaluation may be made that the drowsiness level is low when the Lyapunov exponent is large and the drowsiness level is high when the Lyapunov exponent is small. Thus, the vertical axis of FIG. 8 can be regarded also as an index of the drowsiness level. That is, the drowsiness level is lower when the numerical value on the vertical axis is larger, and the drowsiness level is higher when the numerical value on the vertical axis is smaller. Thus, it is presumed that the numerical value on the vertical axis may be regarded as, for example, a drowsiness exponent showing the drowsiness level.

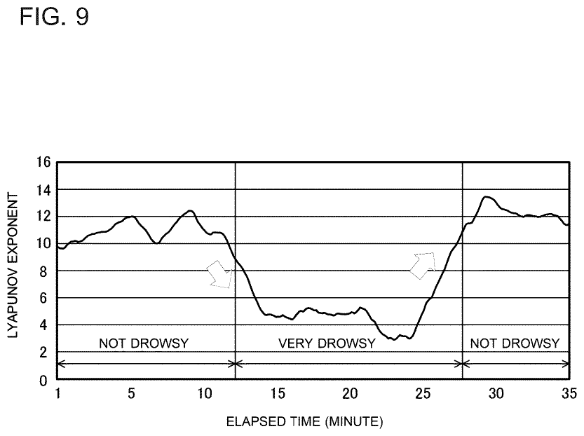

The inventors have installed the Doppler sensor near the working user, and continuously calculated the Lyapunov exponents based on results of continuous measurement of the pulse wave of the user. The inventors have calculated the Lyapunov exponents in the user's subjective report that “he/she had drowsiness” that occurred several times a day, for example, after lunch, and in the user's subjective report that “he/she had no drowsiness.” FIG. 9 illustrates results of calculation of the Lyapunov exponents.

FIG. 9 is a diagram illustrating an example of changes in the Lyapunov exponent during work. The horizontal axis represents elapsed time (minute), and the vertical axis represents the Lyapunov exponent at that time. FIG. 9 also illustrates results of the drowsiness level in the user's subjective report. FIG. 9 is a graph obtained by plotting the results of the Lyapunov exponent calculated at intervals of 1 minute. FIG. 9 demonstrates changes in the Lyapunov exponent in response to changes in the user's subjective report on the drowsiness level, showing a “not drowsy” state, a “very drowsy” state, and a “not drowsy” state in this order. The elapsed times of the “not drowsy,” “very drowsy,” and “not drowsy” states are continuous times of about 12 minutes, 15 minutes, and 7 minutes, respectively.

FIG. 9 demonstrates that, in comparison between the “not drowsy” state and the “very drowsy” state, the Lyapunov exponent is large in the “not drowsy” state and small in the “very drowsy” state. That is, the value of the Lyapunov exponent is large when the drowsiness level is low, and is small when the drowsiness level is high. The Lyapunov exponent gradually decreases when the “not drowsy” state changes to the “very drowsy” state. The Lyapunov exponent gradually increases when the “very drowsy” state changes to the “not drowsy” state.

The above measurement results demonstrate that the Lyapunov exponent and the drowsiness level have a correlation. When the drowsiness level increases during work, the work efficiency decreases. Therefore, the measurement results can be utilized by, for example, being displayed on the display unit 107 to alert the user and prompt the user to take a rest depending on the drowsiness level.

The values of the Lyapunov exponent on the vertical axis of FIG. 9 are obtained by calculation using the drowsiness level estimation device 1 temporarily as an experimental device. FIG. 9 demonstrates that the Lyapunov exponents are obtained at intervals of 1 minute by the drowsiness level estimation device 1. FIG. 9 also demonstrates that the process of calculating the Lyapunov exponents from the pulse wave can be performed in 1 minute that is generally a short time. Although FIG. 9 illustrates the experiment results obtained using the drowsiness level estimation device 1, FIG. 9 illustrates the experiment results of measurement of the correlation between the Lyapunov exponent and the drowsiness level, and may be created using a device dedicated to experiment instead of the drowsiness level estimation device 1.

The inventors have installed the Doppler sensor near the user before and after sleep, and continuously measured the Lyapunov exponents. The result of comparison between the Lyapunov exponent in the subjective report that “the user had drowsiness” before the sleep and the Lyapunov exponent in the subjective report that “the user had no drowsiness” demonstrates that the Lyapunov exponent is smaller, even before the sleep, when the user had drowsiness than when the user had no drowsiness. That is, the drowsiness level and the Lyapunov exponent before the sleep have a correlation. When the drowsiness level increases before the sleep, the user can smoothly fall asleep. Therefore, the measurement results can be utilized by, for example, being displayed on the display unit 107 to notify the user and prompt the user to go to bed depending on the drowsiness level.

As described above, the Lyapunov exponent and the drowsiness level have the direct correlation, and the Lyapunov exponent may be handled also as a mental index that is the drowsiness level. Specifically, evaluation can be made that the drowsiness level increases as the Lyapunov exponent decreases and the drowsiness level decreases as the Lyapunov exponent increases. This method does not require linking to a large amount of past data and storage of complex data, and the drowsiness level can be evaluated easily in real time.

In view of the above relationship, the drowsiness level estimation device 1 estimates the drowsiness level based on the Lyapunov exponent.

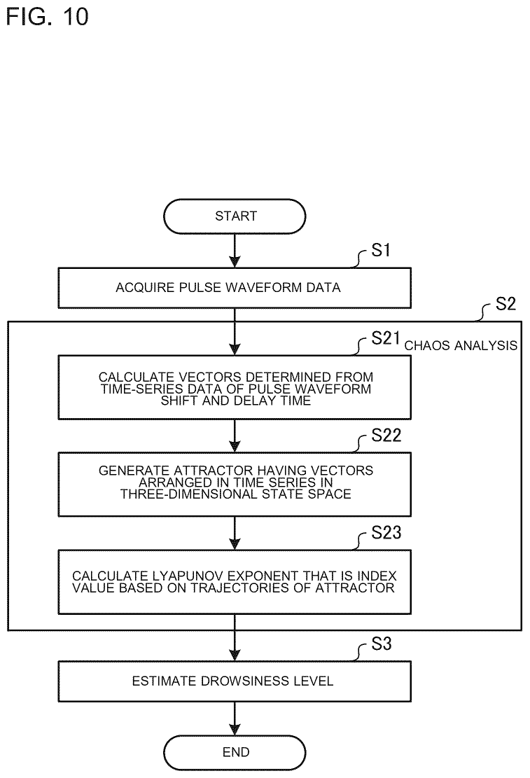

FIG. 10 is a flowchart of the drowsiness level estimation process in the drowsiness level estimation device 1 according to Embodiment 1. The drowsiness level estimation device 1 performs a step of acquiring pulse waveform data (Step S1), and a step of performing chaos analysis based on the pulse waveform data (Step S2). As described above, the chaos analysis step includes three steps (Steps S21 to S23). The first step is a step of calculating vectors determined from time-series data of a pulse waveform shift and the preset delay time (Step S21). The second step is a step of generating an attractor having the vectors arranged in time series in a three-dimensional state space (Step S22). The third step is a step of calculating a Lyapunov exponent that is an index value based on trajectories of the attractor (Step S23).

The analysis unit 103 estimates a drowsiness level based on the Lyapunov exponent (Step S3), and outputs drowsiness level information showing the estimation result. The analysis unit 103 estimates that the drowsiness level increases as the Lyapunov exponent decreases and the drowsiness level decreases as the Lyapunov exponent increases. The analysis unit 103 may output the Lyapunov exponent as the drowsiness level information, or may, for example, convert the Lyapunov exponent into a grade value (e.g., 1 to 10) showing the drowsiness level and output the grade value. The grade value is, for example, a numerical value showing the drowsiness level in ascending order along with the change in the numerical value in ascending order. For example, the analysis unit 103 may hold the Lyapunov exponent during rest, and output, as a numerical value showing the drowsiness level, a numerical value of the Lyapunov exponent obtained by analysis in the analysis unit 103 and expressed in percentage (%) of the Lyapunov exponent during the rest. The analysis unit 103 need not always output the drowsiness level as a numerical value as described above, and may output, for example, a grade image. The grade image refers to, for example, a facial expression image that varies depending on the grade of the drowsiness level. As described above, the analysis unit 103 outputs the Lyapunov exponent as the drowsiness level information showing the drowsiness level.

The drowsiness level information output from the analysis unit 103 is input to the display unit 107 via the cloud unit 106 and displayed on the display unit 107. Since the drowsiness level estimation device 1 visualizes the drowsiness level information by outputting it from the analysis unit 103, the user can grasp the drowsiness level. The drowsiness level information output from the analysis unit 103 may be accumulated in the data collection unit 108 via the cloud unit 106. The drowsiness level information output from the analysis unit 103 may directly be input to and displayed on the display unit 107 without intermediation of the cloud unit 106. The drowsiness level information output from the analysis unit 103 may be input to the control detail determination unit 104 and used to determine details of control on the apparatus including the drowsiness level estimation device 1.

The drowsiness level information may be information showing a current drowsiness level, time-series drowsiness levels, or both of them.

As described above, when the drowsiness level in the subjective report is high, the brain activity level is low and the Lyapunov exponent is low. Thus, the drowsiness level estimation device 1 is suitable when the analysis unit 103 outputs information showing that the drowsiness level increases as the Lyapunov exponent decreases.

The drowsiness level can be regarded also as an index inverse to the concentration level. Therefore, the analysis unit 103 may output concentration level information instead of the drowsiness level information. Thus, the user can view the concentration level.

Although the description has been made that the drowsiness level estimation device 1 can output the drowsiness level information from the index value based on the brain activity amount estimation result, the term or expression “drowsiness level” is not limitative, and any other term or expression may be used as long as the human emotion or mentality and the behaviors with high brain activity levels have the same meanings. For example, the “drowsiness level” may be rephrased as “boredom level” or “laziness level.” Thus, the information obtained from the brain activity amount may be expressed as “boredom level” or “laziness level” instead of “drowsiness level.”

As described above, the drowsiness level estimation device 1 of Embodiment 1 includes the non-contact Doppler sensor 10 that detects a pulse wave in a wide range of the body surface of the user using a radio wave, and the analysis unit 103 that analyzes the pulse wave detected by the Doppler sensor 10. The analysis unit 103 generates an index value by digitizing the pulse wave based on the chaos analysis in which the source of analysis is a pulse waveform shift that is a time-series shift of the waveform of the pulse wave, and estimates the drowsiness level of the user based on the index value.

As described above, the drowsiness level estimation device 1 can estimate the drowsiness level based on the index value generated based on the time-series shift of the waveform of the pulse wave. The pulse wave is vital data related to heart pulsation and furthermore activities in the brain nervous system. The drowsiness level estimation device 1 estimates the drowsiness level based on the index value calculated based on the pulse wave. Therefore, the drowsiness level can be estimated with higher accuracy than in the conventional estimation method based on an action of resting the chin on the hand and other actions.

The drowsiness level estimation device 1 uses the Doppler sensor 10 as the sensor that detects the pulse wave of the user. Description is made about the superiority of the drowsiness level estimation device 1 that estimates the drowsiness level based on the pulse wave detected using the Doppler sensor 10 instead of the contact pulse wave sensor.

Technologies disclosed in Japanese Unexamined Patent Application Publication Nos. 4-208136, 2009-195384, and 2015-16273 are technologies of detecting the psychosomatic state of a user using the pulse wave of the user. In each of the known technologies, the pulse wave is detected using a contact pulse wave sensor. Specifically, in each of the known technologies, the pulse wave sensor attached to the fingertip is used to measure the pulse wave by causing, for example, a photodiode to detect movement of hemoglobin flowing through a capillary vessel.

The contact pulse wave sensor captures the pulsing oscillation as a wave like the Doppler sensor 10, but the attachment portion is limited to, for example, the fingertip located away from the brain that is a main part of the brain activity to be used for the drowsiness level estimation. Therefore, the information acquired by the pulse wave sensor is limited compared with the information acquired by the Doppler sensor 10, and hardly shows the effect of the brain activity that is the central nerve activity.

The drowsiness level estimation device 1 detects the pulsing oscillation of the blood vessels using the non-contact Doppler sensor 10 with the radio wave at 24 GHz or more. The Doppler sensor 10 can detect, using the radio wave, not only the pulsing oscillation of the body surface but also head information in the range that the radio wave reaches or the oscillation of the blood vessels in the body as well as the body surface. The Doppler sensor 10 can acquire pulse wave information of the entire body in the radio wave area. Therefore, the drowsiness level estimation device 1 obtains more information on the autonomic nerve or the central nerve, thereby being advantageous in that the correlation accuracy in the analysis of the change in the pulse waveform increases.

As described above, the drowsiness level estimation device 1 can acquire not only the information on the finger part located away from the brain but also the pulse wave information of the entire body including the head, shoulders, or chest using the Doppler sensor 10. With the Doppler sensor 10, the drowsiness level estimation device 1 can measure the pulse wave affected more greatly by the brain activity that is the central nerve activity and furthermore the drowsiness level. As a result, the drowsiness level can be estimated with high accuracy.

The Doppler sensor 10 can detect fine oscillation of the entire body caused by heart pulsation. The Doppler sensor 10 internally mixes a transmitted signal with a received signal, extracts a variation component caused by the Doppler effect, and generates a pulse wave of an IQ signal. To be exact, the pulse wave information generated by the Doppler sensor 10 is pulse wave information generated using the radio IQ signal unlike a plethysmogram obtained by the pulse wave sensor attached to the fingertip.

The above technologies of Japanese Unexamined Patent Application Publication Nos. 4-208136, 2009-195384, and 2015-16273 use chaos analysis of the pulse wave to detect the psychosomatic state of the user. However, the known technologies are different, in terms of finally obtained information, from the drowsiness level estimation device 1 that estimates the drowsiness level of the user.

In Japanese Unexamined Patent Application Publication No. 4-208136, the body or psychological state of a test subject is obtained, such as “relaxed,” “reading book,” “reading comic book,” or “viewing beautiful picture.” In Japanese Unexamined Patent Application Publication No. 2009-195384, a fatigue level is obtained as the level of human fatigue. In Japanese Unexamined Patent Application Publication No. 2015-16273, a mental balance of a test subject is obtained. For example, information is obtained as to which of “ideal zone,” “too nervous,” “depressed,” “too lazy,” “on instinct,” and “semi-ideal zone” corresponds to the mental state of the test subject.

As described above, the known examples are different from the technology of estimating the drowsiness level of the user because the finally obtained information is different.

The chaos analysis is a process of sequentially performing a step of calculating vectors determined from time-series data of a pulse waveform shift and a preset delay time, a step of generating an attractor having the vectors arranged in time series in a multidimensional state space such as a three-dimensional or higher-dimensional state space, and a step of calculating, as a Lyapunov exponent that is an index value, an enlargement factor obtained by giving a hypersphere as an initial state to trajectories of the attractor and repeating an operation of stretching the hypersphere at intervals of the preset slide time based on the trajectories so that the hypersphere turns into an ellipse.

In this manner, the drowsiness level estimation device 1 can calculate the Lyapunov exponent that is the index value by the chaos analysis.

The method that is used by the analysis unit 103 to calculate the Lyapunov exponent from the attractor in the chaos analysis of the pulse wave is greatly different from a WORF method that is used in the above known examples of Japanese Unexamined Patent Application Publication Nos. 4-208136, 2009-195384, and 2015-16273.

In Japanese Unexamined Patent Application Publication No. 4-208136, a search is made for a next point from a start point of finally obtained two-dimensional data, and the Lyapunov exponent is calculated from the ratio between time and a movement distance. Japanese Unexamined Patent Application Publication No. 4-208136 uses the WORF method in which the Lyapunov exponent is obtained by repeatedly averaging, in the entire attractor, the Lyapunov exponents each calculated from the movement distance from point to point.

In Japanese Unexamined Patent Application Publication No. 2009-195384, an attractor is reconfigured in a predetermined time range on continuous data calculation values, and the time range is slid by 1 second each time so that the value of the maximum Lyapunov exponent is plotted every 1 second. Thus, the WORF method is similarly used in Japanese Unexamined Patent Application Publication No. 2009-195384.

In Japanese Unexamined Patent Application Publication No. 2015-16273, the WORF method is similarly used for a mental flexibility detection process.

The WORF method used in the known examples is briefly described below.

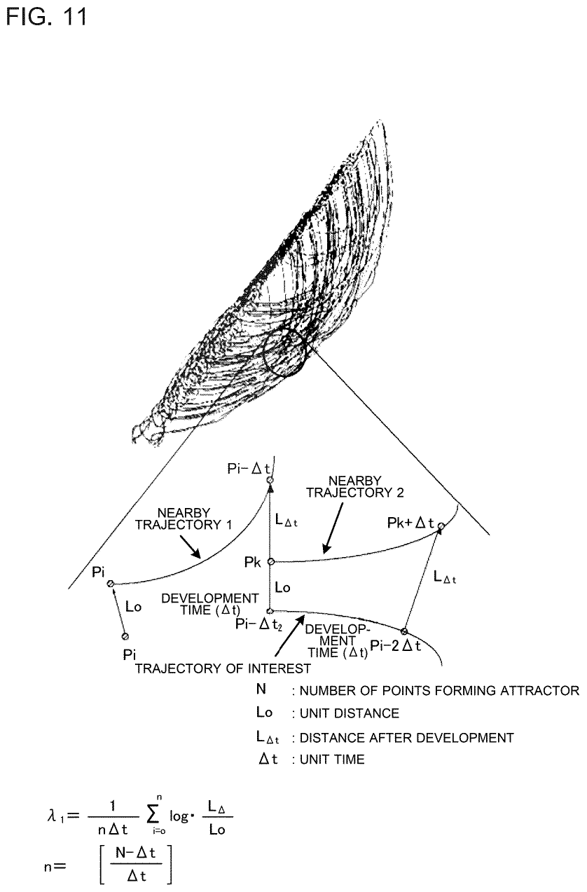

FIG. 11 is an explanatory diagram of the WORF method used in the known examples. The WORF method described in Alan WOLF et al., DETERMINING LYAPUNOV EXPONENTS FROM A TIME SERIES, Physica 16D (1985) 285-317 is described in detail.

The WORF method is simpler than the Lyapunov exponent calculation method of the analysis unit 103. A search is made for a point Pj located at a unit distance L₀ from a point Pi of interest in a chaos attractor, and the logarithm of the ratio between L₀ and a distance L_{Δt} between the points after a unit time Δt is calculated. In the WORF method, the logarithm calculation process is performed on the entire chaos attractor with Pi shifted, and a numerical value obtained by simply averaging the plurality of calculated logarithms is set as a Lyapunov exponent λ1. If the analysis target has periodicity, λ1 is 0, and if it is chaotic, λ1 is a positive value. In the WORF method, the Lyapunov exponent is calculated after the attractor is converted into a two-dimensional image. In the WORF method, an increase or decrease in the distance between the two points is calculated, and the information or behavior in the dynamical system is simple.

The Lyapunov exponent calculation method of the analysis unit 103 is as described above. An enlargement factor is calculated from a temporal change in multidimensional directions in a microsphere (hypersphere) given as an initial state on trajectories in multiple dimensions that are three or more dimensions, and the extension or contraction of the three-dimensional object is calculated. Since the enlargement factor is calculated from how the hypersphere in the multiple dimensions develops continuously at each time, a large amount of information is obtained and the calculation accuracy increases. This point is the large difference from WORF.

In the known technologies, the Lyapunov exponent obtained by the WOLF method has a poor correlation with the brain activity and furthermore the drowsiness level. According to the experiment and research conducted by the inventors, there is no correlation with the brain activity.

With the above method in which the analysis unit 103 performs the Lyapunov exponentiation by giving the microsphere (hypersphere) to the multidimensional chaos dynamical system, the states with different brain activity amounts can be measured promptly, and the Lyapunov exponent can be obtained as an estimation index having a high linearity and a strong correlation with the brain activity amount and furthermore the drowsiness level.

The known examples are different from the present disclosure in terms of the relationship between the Lyapunov exponent and the brain activity obtained based on the user test results. Japanese Unexamined Patent Application Publication No. 4-208136 describes that the Lyapunov exponent decreases as the concentration increases, and the Lyapunov exponent decreases as the information processing in the brain is more active. That is, in Japanese Unexamined Patent Application Publication No. 4-208136, the Lyapunov exponent is a value that decreases as the brain activity level increases. The Lyapunov exponent that is the index value obtained by the analysis unit 103 is a value that increases when the brain activity level increases, in other words, the drowsiness level decreases. The meaning of the Lyapunov exponent as the index value is completely opposite to that in the known example.

Japanese Unexamined Patent Application Publication No. 4-208136 describes that the attractor is output to a two-dimensional screen and the user is more relaxed as the size of the attractor decreases. In Japanese Unexamined Patent Application Publication No. 4-208136, the attractor is the two-dimensional attractor, and therefore sparseness and denseness are described about a local structure of the spiral of the attractor. When the concentration increases, the local structure changes from sparse to dense. However, the sparse/dense relationship of the attractor has no relationship with the Lyapunov exponent obtained by the analysis unit 103.

Japanese Unexamined Patent Application Publication No. 2015-16273 describes the Lyapunov exponent calculation, but does not describe the user test results related to the Lyapunov exponent and the drowsiness level.

As described above, the above known examples are different from the present disclosure in terms of the relationship between the Lyapunov exponent and the brain activity and furthermore the drowsiness level.

The analysis unit 103 outputs the drowsiness level information showing that the drowsiness level decreases as the index value increases, and that the drowsiness level increases as the index value decreases. The drowsiness level information is information showing a current drowsiness level, time-series drowsiness levels, or both of them.

Thus, the user can grasp the drowsiness level based on the drowsiness level information.

### Embodiment 2

The drowsiness level estimation device 1 of Embodiment 1 estimates the brain activity level of the central nerve by analyzing the pulse waveform fluctuation, and estimates the drowsiness level based only on the brain activity level. A drowsiness level estimation device 1 of Embodiment 2 estimates an autonomic activity level based on a pulse interval fluctuation in addition to the brain activity level, and estimates the drowsiness level based on both the central nerve activity and the autonomic activity. The differences of Embodiment 2 from Embodiment 1 are mainly described below, and the configuration that is not described in Embodiment 2 is similar to that in Embodiment 1.

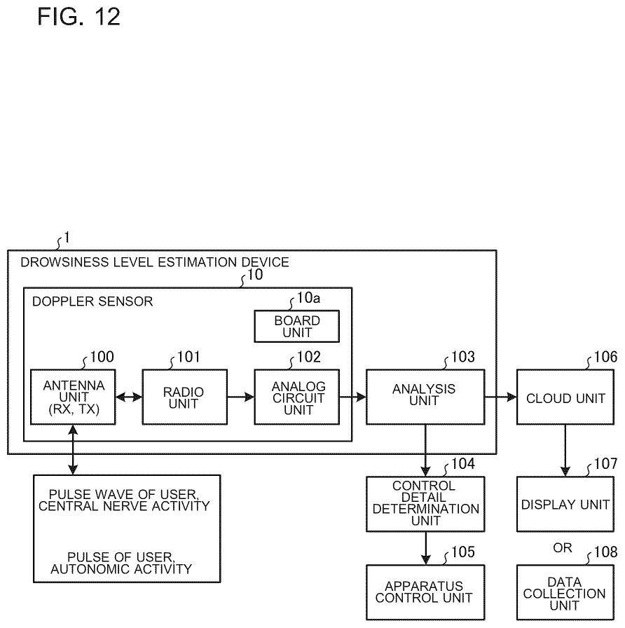

FIG. 12 is a block diagram illustrating the configuration of the drowsiness level estimation device 1 according to Embodiment 2 and the use configuration of the drowsiness level estimation device 1. The Doppler sensor 10 detects the pulse of the user in addition to the pulse wave of the user.

The pulse interval is generally called “R-R Interval (RRI).” The RRI is subjected to frequency conversion into various types of information such as an autonomic balance described later. In vital analysis in which a pulse, blood pressure, and respiration are analyzed, an infinitesimal fluctuation having an extremely low frequency characteristic of about 1 Hz is analyzed unlike body movement analysis or other analysis. Therefore, in the vital analysis, analog detection using a 24-GHz Doppler system is preferable to 60 to 79 GHz that is often used for distance measurement because of high resolution.

The Doppler sensor 10 detects the autonomic balance from a shift of the pulse interval of the user (one-dimensional pattern pulse shift). The autonomic balance is a balance between the sympathetic nerve and the parasympathetic nerve. The autonomic balance is the ratio between a low frequency (LF) and a high frequency (HF), and is calculated by LF/HF. LF shows a sympathetic nerve activity, and HF shows a parasympathetic nerve activity. The sympathetic nerve is dominant during the daytime or in an active state, and the parasympathetic nerve is dominant during the nighttime or in a calm state.

LF is obtained by an integrated value of powers in a low frequency band of, for example, 0.05 Hz to 0.15 Hz on a characteristic curve. HF is obtained by an integrated value of powers in a high frequency band of, for example, 0.15 Hz to 0.40 Hz on the characteristic curve. The characteristic curve is obtained by frequency expansion on time-series pulse intervals, and is plotted on coordinate axes including the horizontal axis representing frequency and the vertical axis representing power.

Since the autonomic balance is LF/HF, when LF is relatively large, the sympathetic nerve is dominant, and an excited or active state can be estimated. When LF is relatively small, the parasympathetic nerve is dominant, and a relaxed state can be estimated. When the numerical value of the autonomic balance is large, the excited state can be estimated. When the numerical value of the autonomic balance is small, the relaxed or comfortable state can be estimated. When the human body is excited or active, the activity level in the autonomic nervous system is high, and the value of the autonomic balance is large. When the human body is relaxed, the activity level in the autonomic nervous system is low, and the value of the autonomic balance is small. Thus, the autonomic balance can be used as an index showing the autonomic activity level.

The correspondence relationship among the autonomic balance, the autonomic activity level, and the state of the user is as follows. That is, when the numerical value of the autonomic balance is small, the autonomic activity level is low, and the user is relaxed or comfortable. When the numerical value of the autonomic balance is large, the autonomic activity level is high, and the user is excited or active.

In this manner, the Doppler sensor 10 can detect the autonomic activity level based on the autonomic balance. As described above, the Doppler sensor 10 can detect the brain activity level in the central nervous system based on the pulse wave. That is, the Doppler sensor 10 alone can detect the autonomic activity and the central nerve activity. The pulse that can be measured by the Doppler sensor 10 refers to a pulse rate or a pulsing motion, and includes the magnitude of the pulse rate or a time-series increase or decrease in the pulse rate, the magnitude of the pulse interval or a time-series increase or decrease in the pulse interval, the magnitude of LF or a time-series increase or decrease in LF, and the magnitude of LF/HF or a time-series increase or decrease in LF/HF.

The drowsiness level estimation device 1 of Embodiment 2 estimates the drowsiness level from both the central nerve activity and the autonomic activity. The concept of drowsiness level estimation from both the central nerve activity and the autonomic activity is described below.

First, the inventors have found that the autonomic activity level is related to the accuracy of the drowsiness level. The inventors have conducted experiment in which, during a predetermined time, the pulse of the user is measured and subjective reports are received from the user as to whether the user is drowsy or not. As a result, the inventors have found that the pulse rate of a plurality of users who is drowsy is lower by 4 beats per minute on average than the pulse rate of the plurality of users who is not drowsy. Thus, the pulse rate affects the drowsiness level estimation.

Although the drowsiness level can be estimated only by the brain activity level in the central nervous system as described above, the inventors have found that the estimation accuracy can be increased when the drowsiness level is estimated by the autonomic activity level in combination. Although the drowsiness level increases as the brain activity level decreases, the inventors have found through a test that, when the autonomic activity level is lower while the brain activity level is low, the user is relaxed and the drowsiness level is higher. Although the drowsiness level decreases as the brain activity level increases, the inventors have found through a test that, when the autonomic activity level is higher while the brain activity level is high, the user is excited or active and the drowsiness level is lower.

The inventors have found through the tests that the accuracy is improved for the result of estimation that the drowsiness level increases as the autonomic activity level decreases, and that the accuracy is improved for the result of estimation that the drowsiness level decreases as the autonomic activity level increases. That is, the inventors have found a new advantageous effect in that the accuracy is improved for both the high-drowsiness state and the low-drowsiness state by the combination of the two factors that are the brain activity level in the central nervous system and the autonomic activity level.

Using a simple example, description is made about the fact that the drowsiness level is related not only to the brain activity level in the central nervous system but also to the autonomic activity level. While the human autonomic nerve is working all day long, the human is highly relaxed or comfortable when he/she has drowsiness, in particular, falls into a doze. Therefore, it is considered that the drowsiness level can be estimated with higher accuracy when the autonomic activity as well as the brain activity in the central nervous system is evaluated. Thus, there is an advantageous effect in that the drowsiness level can be estimated with high accuracy using both the brain activity in the central nervous system and the autonomic activity.

The drowsiness level is affected by the central nerve activity more greatly than the autonomic activity. In other words, the brain activity that is the central nerve activity has a higher contribution factor to the drowsiness level than the autonomic activity. Therefore, in the drowsiness level estimation, the brain activity level may be used with a contribution factor of at least 50% or more and 90% or less. The brain activity level is preferably used with a contribution factor of 60% or more and 80% or less. When the contribution factor of the brain activity level is outside the above range, the accuracy of the estimated drowsiness level may decrease. Thus, the contribution factor of the brain activity level is preferably 60% or more and 80% or less.

In the drowsiness level estimation, the autonomic activity level may be used with a lower contribution factor than that of the brain activity level. The autonomic activity level is preferably used with a contribution factor of 20% or more and 40% or less. When the contribution factor of the autonomic activity level is outside the above range, the accuracy of the estimated drowsiness level may decrease. Thus, the contribution factor of the autonomic activity is preferably 20% or more and 40% or less. The contribution factor of the brain activity level to the drowsiness level is hereinafter referred to as “first contribution factor.” The contribution factor of the autonomic activity level to the drowsiness level is hereinafter referred to as “second contribution factor.” The first contribution factor shows how greatly the brain activity level affects the drowsiness level. The second contribution factor shows how greatly the autonomic activity level affects the drowsiness level.

An example of the drowsiness level estimation using the contribution factors is described below. When the first contribution factor is 70% and the second contribution factor is 30%, the drowsiness level is calculated by “drowsiness level=0.7×brain activity level+0.3×autonomic activity level.” For example, it is assumed that the drowsiness level is expressed by a grade value of 1 to 100 and the drowsiness level increases as the value increases. It is also assumed that the brain activity level and the autonomic activity level are expressed by grade values of 1 to 100. Since the numerical value of the drowsiness level increases when the drowsiness level increases, larger numerical values are assigned to the brain activity level and the autonomic activity level for use in the above calculation formula as the drowsiness level increases. That is, the brain activity level has a larger value as the brain activity level decreases, and the autonomic activity level has a larger value as the autonomic activity level decreases.

It should be noted that the magnitude relationship of the numerical value of the autonomic activity level for use in the above calculation formula is opposite to the magnitude relationship of the autonomic activity level based on LF/HF showing the autonomic balance. That is, the autonomic activity level based on LF/HF has a larger value as the autonomic activity level increases, and the magnitude relationship is opposite to that of the numerical value of the autonomic activity level for use in the above calculation formula. The magnitude relationship of the numerical value of the autonomic activity level for use in the above calculation formula is used only in the drowsiness level calculation using the contribution factors. In the following description, the autonomic activity level has the magnitude relationship based on LF/HF.

When the brain activity level and the autonomic activity level are expressed by the above grade values, the brain activity level is 100, and the autonomic activity level is 100, the drowsiness level is the maximum value of 100 based on the above formula. When the brain activity level is 80 and the autonomic activity level is 50, the drowsiness level is 71.

The concept of drowsiness level estimation using both the brain activity level and the autonomic activity level has been described clearly above. The description now returns to FIG. 12.

The drowsiness level estimation device 1 detects the pulse wave and the pulse of the user with the Doppler sensor 10. The analysis unit 103 estimates the brain activity level in the central nervous system by analyzing the pulse wave detected by the Doppler sensor 10, and estimates the activity level in the autonomic nervous system from the pulse. The analysis unit 103 has the first contribution factor and the second contribution factor. The first contribution factor is larger than the second contribution factor. The second contribution factor is smaller than the first contribution factor. As described above, the first contribution factor is preferably 60% or more and 80% or less, and the second contribution factor is preferably 20% or more and 40% or less. The analysis unit 103 estimates the drowsiness level in the manner described above based on the brain activity level, the autonomic activity level, the first contribution factor, and the second contribution factor. Then, the analysis unit 103 outputs drowsiness level information showing the estimated drowsiness level.

The drowsiness level information output from the analysis unit 103 is input to the display unit 107 via the cloud unit 106 and displayed on the display unit 107. Since the drowsiness level estimation device 1 visualizes the drowsiness level information by outputting it from the analysis unit 103, the user can grasp the drowsiness level. The drowsiness level information output from the analysis unit 103 may be accumulated in the data collection unit 108 via the cloud unit 106. The drowsiness level information output from the analysis unit 103 may directly be input to and displayed on the display unit 107 without intermediation of the cloud unit 106. The drowsiness level information output from the analysis unit 103 may be input to the control detail determination unit 104 and used to determine details of control on the apparatus including the drowsiness level estimation device 1.

The human autonomic nerve is working all day long. During work or study mainly in the daytime, the sympathetic nerve in the autonomic nervous system called “good stress” is highly active. When the sympathetic nerve in the autonomic nervous system is highly active, the human excites the brain, dilates the trachea, increases the heart rate, constricts the blood vessels, increases the blood pressure, suppresses gastrointestinal motility, and promotes sweating. Thus, the highly active sympathetic nerve in the autonomic nervous system is good for work and study. That is, when the sympathetic nerve of LF is highly active or the ratio expressed by LF/HF about the autonomic balance is high, the user is in a good condition for work and study. When “drowsiness level is low” and “excited or active emotion is high,” the user can make progress.

That is, when the drowsiness level is low and the ratio about the autonomic balance is high, evaluation can be made that the user is in an optimum condition for work and study, in other words, the user's work efficiency is high. Thus, the analysis unit 103 may output work efficiency information showing that the user's work efficiency is high when the brain activity level is lower than a preset first threshold and the autonomic activity level is higher than a preset second threshold. In this manner, the analysis unit 103 may output not only the drowsiness level but also other information determined from the brain activity level and the autonomic activity level.

When the sympathetic nerve in the autonomic nervous system is less active, the human calms the brain, narrows the trachea, reduces the heart rate, dilates the blood vessels, reduces the blood pressure, activates gastrointestinal motility, and suppresses sweating. Thus, the less active sympathetic nerve in the autonomic nervous system leads to relaxation. That is, when the sympathetic nerve of LF is less active or the ratio expressed by LF/HF about the autonomic balance is low, the user is relaxed or comfortable. When “drowsiness level is high” and “excited or active emotion is low,” the user is relaxed.

That is, when the drowsiness level is high and the ratio about the autonomic balance is low, evaluation can be made that the user is most relaxed or comfortable, in other words, in a good condition for rest. Thus, the analysis unit 103 may output relaxation level information showing that the user's relaxation level is high when the brain activity level is higher than a preset third threshold and the autonomic activity level is lower than a preset fourth threshold. In this manner, the analysis unit 103 may output not only the drowsiness level but also other information determined from the brain activity level and the autonomic activity level.

As described above, the drowsiness level estimation device 1 of Embodiment 2 can obtain the advantageous effects similar to those of Embodiment 1, and estimate the autonomic activity level by detecting the pulse with the Doppler sensor 10. Therefore, the drowsiness level estimation device 1 can estimate the autonomic activity level in addition to the brain activity level in the central nervous system. The drowsiness level estimation device 1 can estimate the drowsiness level with high accuracy from the brain activity level in the central nervous system, the autonomic activity level, the first contribution factor, and the second contribution factor. Since the central nerve activity and the autonomic activity can be measured by the single Doppler sensor 10, the drowsiness level estimation device 1 can estimate emotions remotely in a short time without the need for other devices compared with a device using an electroencephalograph or an imaging unit.

### Embodiment 3

The drowsiness level estimation devices 1 of Embodiments 1 and 2 each estimate the drowsiness level of the user at a certain time point from the single Lyapunov exponent. A drowsiness level estimation device 1 of Embodiment 3 estimates an average drowsiness level of the user and a change in the drowsiness level in a predetermined section. The differences of Embodiment 3 from Embodiments 1 and 2 are mainly described below, and the configuration that is not described in Embodiment 3 is similar to those in Embodiments 1 and 2.

The drowsiness level estimation device 1 of Embodiment 3 is similar to the drowsiness level estimation devices 1 of Embodiments 1 and 2 in that the analysis unit 103 generates the Lyapunov exponent that is the index value by digitizing the pulse wave based on the chaos analysis in which the source of analysis is the pulse waveform shift that is the time-series shift of the waveform of the pulse wave. The drowsiness level estimation device 1 of Embodiment 3 estimates a drowsiness level of the user at one certain time point (hereinafter referred to as “single-point drowsiness level”) based on the Lyapunov exponent. In the drowsiness level estimation device 1 of Embodiment 3, the analysis unit 103 further estimates an average drowsiness level and a change in the drowsiness level in a predetermined section. The drowsiness level estimation device 1 of Embodiment 3 is different from the drowsiness level estimation devices 1 of Embodiments 1 and 2 in that the analysis unit 103 estimates the average drowsiness level and the change in the drowsiness level in the predetermined section.

As described above, the drowsiness level estimation device 1 can calculate the Lyapunov exponent, estimate the drowsiness level, and output the drowsiness level information at a short time interval of, for example, 1 minute. When the drowsiness level estimation device 1 is provided to an air-conditioning apparatus or other apparatuses and the apparatus changes apparatus control based on the drowsiness level information output from the drowsiness level estimation device 1 at the short time interval, the following problem arises. For example, when the apparatus is an air-conditioning apparatus, the drowsiness level varies at the short time intervals, and the variation in the drowsiness level is reflected directly in, for example, air-conditioning control or alert on a display unit, the change in the air-conditioning control or the alert on the display unit are frequently performed in a short time, and the user may be dissatisfied.

In view of the control to be performed by the apparatus including the drowsiness level estimation device 1, the drowsiness level estimation device 1 calculates and outputs, in addition to the single-point drowsiness level, a first-order short section drowsiness level B obtained by averaging time-series data of the single-point drowsiness levels, and a second-order short section drowsiness level C obtained by averaging time-series data of the first-order short section drowsiness levels B. That is, the drowsiness level estimation device 1 calculates an average drowsiness level of the user in a predetermined section, and outputs drowsiness level information for each predetermined section. Thus, the apparatus including the drowsiness level estimation device 1 performs the apparatus control at an appropriate frequency, and the number of times of unnecessary apparatus control can be reduced.

The drowsiness level estimation device 1 can digitize the drowsiness level at a certain time point or in a predetermined section. Since the magnitude of the numerical value differs depending on users, estimation as to whether the drowsiness level increases or decreases is further required in view of versatility of an apparatus for a large number of unspecified users. When the increase or decrease in the drowsiness level is determined, the apparatus including the drowsiness level estimation device 1 can perform appropriate apparatus control and issue an alert at an appropriate timing using an application screen, sound, or light based on the increase or decrease in the drowsiness level. Therefore, the drowsiness level estimation device 1 of Embodiment 3 can also estimate a change in the drowsiness level as detailed below.

A specific configuration is described below. The drowsiness level estimation device 1 of Embodiment 3 is different from the drowsiness level estimation devices 1 of Embodiments 1 and 2 in terms of the configuration of the analysis unit 103.

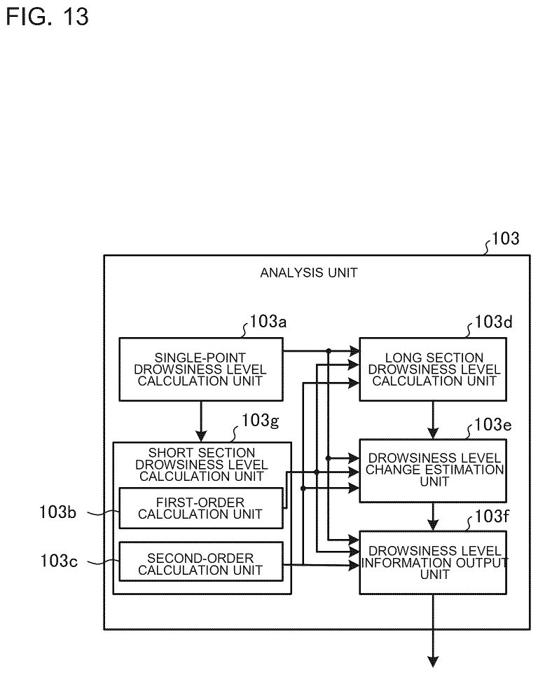

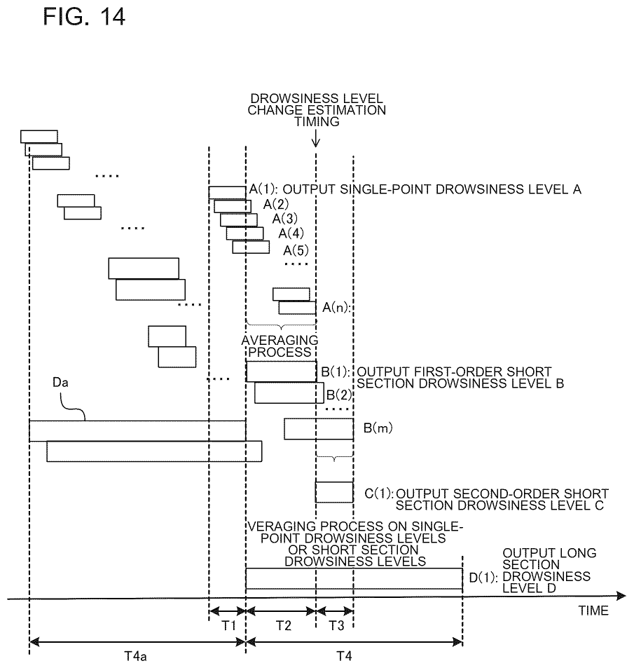

FIG. 13 is a block diagram of the analysis unit 103 of the drowsiness level estimation device 1 according to Embodiment 3. The analysis unit 103 includes a single-point drowsiness level calculation unit 103a, a short section drowsiness level calculation unit 103g, a long section drowsiness level calculation unit 103d, a drowsiness level change estimation unit 103e, and a drowsiness level information output unit 103f. The short section drowsiness level calculation unit 103g calculates a short section drowsiness level by performing an averaging process on time-series data of the single-point drowsiness levels in a short section, and includes a first-order calculation unit 103b that calculates the first-order short section drowsiness level B, and a second-order calculation unit 103c that calculates the second-order short section drowsiness level C. Those calculation units are each functionally configured by a CPU of the analysis unit 103 and a control program. Operations of the calculation units, the first-order short section drowsiness level B, the second-order short section drowsiness level C, and a long section drowsiness level D are described below with reference to FIGS. 13 and 14.

FIG. 14 is a conceptual diagram of the first-order short section drowsiness level B, the second-order short section drowsiness level C, and the long section drowsiness level D calculated by the drowsiness level estimation device 1 according to Embodiment 3. The single-point drowsiness level calculation unit 103a calculates a Lyapunov exponent by the process including the first to third steps described in Embodiment 1, estimates a single-point drowsiness level A based on the Lyapunov exponent, and outputs it. Specifically, the single-point drowsiness level calculation unit 103a first generates an attractor based on vectors X(0) to X(n), and calculates a Lyapunov exponent based on trajectories of the attractor. Then, the single-point drowsiness level calculation unit 103a calculates a single-point drowsiness level A by indexation of a drowsiness level at one time point based on the calculated Lyapunov exponent, and outputs it. The Lyapunov exponent used to calculate the single-point drowsiness level A may be a raw value of the Lyapunov exponent, or may be a value obtained by normalizing the raw value of the Lyapunov exponent by percentage or grades. In any case, the single-point drowsiness level calculation unit 103a calculates the single-point drowsiness level A at one time point based on the Lyapunov exponent and outputs it.

In one example, when a time T1 that is the output window length is 60 seconds and a sampling time interval is 1 second, the single-point drowsiness level calculation unit 103a generates an attractor from 60 pieces of time-series data X(0) to X(60) obtained in 60 seconds, and outputs one single-point drowsiness level A after an elapse of 60 seconds from the start of measurement. In another example, when the time T1 is 60 seconds and the sampling time interval is 2 seconds, the single-point drowsiness level calculation unit 103a generates an attractor from 30 pieces of time-series data obtained in 60 seconds, and outputs one single-point drowsiness level A after an elapse of 60 seconds from the start of measurement.

The time T1 and the sampling time interval can be selected as appropriate depending on the accuracy, calculation time, and memory size. Since the human drowsiness level changes even in about 1 minute, however, the time T1 is optimally 1 minute or less. When the time T1 is conversely a long time such as 5 minutes, the drowsiness level increases, decreases, or changes in different directions during measurement, and the attractor cannot be generated accurately. Thus, the long time T1 is inappropriate. When the time T1 is a long time such as 5 minutes, there is a disadvantage in that the single-point drowsiness level A is smoothed during the long time and the midway drowsiness level change is hardly observed. This leads to a decrease in the estimation accuracy. Thus, the time T1 is preferably less than 5 minutes.

The single-point drowsiness level calculation unit 103a repeats the process of outputting the single-point drowsiness level A during the measurement using the Doppler sensor 10. The single-point drowsiness level calculation unit 103a uses time-series vector data of the vectors X used to calculate the single-point drowsiness level A while shifting the time-series vector data by the sampling time interval each time. The description is made below under the assumption that the sampling time interval of each calculation unit of the analysis unit 103 is 1 second.

A specific example in which the time T1 is 60 seconds is described. In this case, the single-point drowsiness level calculation unit 103a generates an attractor based on 60 pieces of time-series vector data X(1) to X(60) obtained in 60 seconds from the start of measurement, calculates a single-point drowsiness level A(1) firstly, and outputs it. To obtain a single-point drowsiness level A(2) secondly, the single-point drowsiness level calculation unit 103a uses time-series vector data X(2) to X(61) by shifting the time-series vector data. That is, the single-point drowsiness level calculation unit 103a generates an attractor based on the time-series vector data X(2) to X(61), calculates the single-point drowsiness level A(2), and outputs it. Similarly, the single-point drowsiness level calculation unit 103a generates an attractor based on time-series vector data X(3) to X(62), and outputs a single-point drowsiness level A(3). After the single-point drowsiness level A(1) is output firstly, the single-point drowsiness level calculation unit 103a repeats the process of outputting the single-point drowsiness level A every 1 second until the end of measurement.

The first-order calculation unit 103b acquires time-series data of the single-point drowsiness levels A output from the single-point drowsiness level calculation unit 103a. The first-order calculation unit 103b calculates a first-order short section drowsiness level B by performing an averaging process such as a moving average process on the time-series data of the single-point drowsiness levels A acquired in a short section. In FIG. 14, the short section for the first-order calculation unit 103b is a time T2. That is, the first-order calculation unit 103b calculates the first-order short section drowsiness level B by averaging the plurality of single-point drowsiness levels A within the time T2. The first-order calculation unit 103b outputs the calculated first-order short section drowsiness level B. The first-order calculation unit 103b uses the time-series data of the single-point drowsiness levels A used to calculate the first-order short section drowsiness level B while shifting the time-series data by the sampling time interval each time.

A specific example in which the time T2 is 90 seconds, that is, “n” in FIG. 14 is 90 is described. In this case, the first-order calculation unit 103b calculates a first-order short section drowsiness level B(1) by performing the averaging process on 90 pieces of time-series data A(1), . . . , A(90), and outputs it. To calculate a first-order short section drowsiness level B(2) secondly, the first-order calculation unit 103b uses time-series data A(2), . . . , A(91) by shifting the time-series data. That is, the first-order calculation unit 103b calculates the first-order short section drowsiness level B(2) using the time-series data A(2), . . . , A(91), and outputs it. After the first-order short section drowsiness level B(1) is output firstly, the first-order calculation unit 103b repeats the process of outputting the first-order short section drowsiness level B every 1 second until the end of measurement.

The first-order short section drowsiness level B is obtained by the averaging process on the time-series data of the single-point drowsiness levels A, and is therefore data that smooths out up-and-down numerical value variations of the single-point drowsiness levels A. That is, the first-order short section drowsiness level B is an index value with which an accurate drowsiness level can be grasped even if the time-series data of the single-point drowsiness levels A has up-and-down variations. By using the first-order short section drowsiness level B as the drowsiness level to perform apparatus control based on the drowsiness level, the apparatus including the drowsiness level estimation device 1 can perform more stable control than in the case where the single-point drowsiness level A is used. Since an increase in the human drowsiness level is observed even in about 1 minute, the time T2 is optimally 1 minute or less similarly to the time T1. When the time T2 is set extremely long, the drowsiness level is excessively smoothed and the midway drowsiness level change may be undetected. Thus, the time T2 is preferably 3 minutes or less.

Various processes such as arithmetical averaging, weighted averaging, geometrical averaging, or harmonic averaging are used as the averaging process. Since the drowsiness level has a time-series tendency, the moving average process is suitable as the averaging process.

Time-series data obtained by calculating the first-order short section drowsiness levels B during a predetermined period is represented by a smooth curve in a graph having a vertical axis representing the first-order short section drowsiness level B, and a drowsiness level that reflects the tendency of the drowsiness level change in each section during the time T2 is finally obtained. This method is effective in a frequently varying event typified by the brain activity or the pulse wave, and in removal of noise superimposed on the input signal.

To further reduce the calculation time or the calculation amount in the drowsiness level estimation, the drowsiness level estimation device 1 preferably uses simple averaging. For example, when the time T2 is 90 seconds, the first-order short section drowsiness level B may be a value obtained by simply averaging the 90 single-point drowsiness levels A, namely A(1), . . . , A(90). In this case, there is an advantage in that the calculation amount is reduced though the accuracy decreases.

The second-order calculation unit 103c acquires time-series data of the first-order short section drowsiness levels B output from the first-order calculation unit 103b. The second-order calculation unit 103c calculates a second-order short section drowsiness level C by further performing the averaging process on the time-series data of the first-order short section drowsiness levels B acquired in a short section, and outputs it. In FIG. 14, the short section for the second-order calculation unit 103c is a time T3. That is, the second-order calculation unit 103c calculates the second-order short section drowsiness level C by averaging the plurality of first-order short section drowsiness levels B within the time T3. The second-order calculation unit 103c outputs the calculated second-order short section drowsiness level C. The second-order calculation unit 103c uses the time-series data of the first-order short section drowsiness levels B used to calculate the second-order short section drowsiness level C while shifting the time-series data by the sampling time interval each time.

A specific example in which the time T3 is 60 seconds, that is, “m” in FIG. 14 is 60 is described. In this case, the second-order calculation unit 103c calculates a second-order short section drowsiness level C(1) by performing the averaging process on 60 pieces of time-series data B(1), . . . , B(60), and outputs it. To calculate a second-order short section drowsiness level C(2) secondly, the second-order calculation unit 103c uses time-series data B(2), . . . , B(61) by shifting the time-series data. That is, the second-order calculation unit 103c calculates the second-order short section drowsiness level C(2) using the time-series data B(2), . . . , B(61), and outputs it. After the second-order short section drowsiness level C(1) is output firstly, the second-order calculation unit 103c repeats the process of outputting the second-order short section drowsiness level C every 1 second until the end of measurement.

The single-point drowsiness level A output from the single-point drowsiness level calculation unit 103a, the first-order short section drowsiness level B output from the first-order calculation unit 103b, and the second-order short section drowsiness level C output from the second-order calculation unit 103c are input to the drowsiness level information output unit 103f. The drowsiness level information output unit 103f may output any one of the single-point drowsiness level A, the first-order short section drowsiness level B, and the second-order short section drowsiness level C as the drowsiness level information, or may output part or all of them.

The second-order short section drowsiness level C is an index value with a stable numerical value or a stable numerical value tendency by further smoothing compared with the first-order short section drowsiness level B. In the second-order short section drowsiness level C, the midway fine drowsiness level increase is hardly observed. Therefore, when outputting any one of the single-point drowsiness level A, the first-order short section drowsiness level B, and the second-order short section drowsiness level C, the drowsiness level information output unit 103f may make selection depending on the frequency, response, and estimated time of control change to be performed by the apparatus including the drowsiness level estimation device 1.

To reduce the calculation time and the calculation amount while increasing the accuracy, the analysis unit 103 may perform the following process. The analysis unit 103 first sets the time T2 to be sufficiently shorter than the time T1, and calculates the first-order short section drowsiness level B by performing a moving average process on the time-series data of the single-point drowsiness levels A within the short time. Thus, the drowsiness level estimation accuracy can be increased compared with the simple averaging. For example, T1 is set to 30 seconds, and T2 is set to 10 seconds to 15 seconds. Then, the analysis unit 103 calculates the second-order short section drowsiness level C by performing the averaging process using simple averaging on the time-series data of the first-order short section drowsiness levels B within the time T3. When T3 is 30 seconds, the drowsiness level estimation device 1 calculates the second-order short section drowsiness level C by simply averaging 30 first-order short section drowsiness levels B. With the simple averaging, the analysis unit 103 has an advantage in that the calculation amount is reduced though the accuracy decreases. Through the above process, the analysis unit 103 can reduce the calculation time and the calculation amount while increasing the accuracy.

The long section drowsiness level calculation unit 103d calculates a long section drowsiness level D. The long section drowsiness level calculation unit 103d calculates the long section drowsiness level D by a method similar to that for the short section drowsiness level using time-series data of the single-point drowsiness levels A or the short section drowsiness levels acquired in a long section that is longer than the above short sections. In FIG. 14, the long section is a time T4. The time T4 is set longer than the time T2 that is the acquisition time for the time-series data used for the calculation of the first-order short section drowsiness level B.

The long section drowsiness level calculation unit 103d calculates the long section drowsiness level D by performing an averaging process such as a moving average process on the time-series data of the single-point drowsiness levels A within the time T4. The long section drowsiness level calculation unit 103d may calculate the long section drowsiness level D by performing the averaging process on the time-series data of the short section drowsiness levels. When the short section drowsiness levels are used for the calculation of the long section drowsiness level D, the long section drowsiness level calculation unit 103d may use either of the first-order short section drowsiness levels B and the second-order short section drowsiness levels C. When the short section drowsiness levels are used in the case where the moving average process is performed as the averaging process, the long section drowsiness level calculation unit 103d can reduce the moving average process time compared with the case where the single-point drowsiness levels A are used, thereby reducing the storage area or the calculation time. The averaging process may be the moving average process, or may be a simple averaging process on a plurality of past short section drowsiness levels when its count is small.

The long section drowsiness level D is defined as an index value showing a normal drowsiness level of the user. The long section drowsiness level D is used for estimation of a change in the drowsiness level by the drowsiness level change estimation unit 103e described later. Estimation as to whether the drowsiness level changes requires a current drowsiness level at the time of estimation and a drowsiness level for comparison. The long section drowsiness level D is used as a target of the comparison. The first-order short section drowsiness level B or the second-order short section drowsiness level C is used as the current drowsiness level at the time of estimation.

As described above, the time T4 is set longer than the time T2. For example, the time T2 is about 1 minute, and is about 5 minutes at the maximum. For example, the time T4 is preferably a value from 3 minutes to 60 minutes. When the time T4 is shorter than 3 minutes, there is no temporal difference from the time T2, and the long section drowsiness level D cannot be differentiated from the first-order short section drowsiness level B. Thus, the comparison is difficult. When the time T4 is equal to or longer than a triple of the time T2, the long section drowsiness level D can be differentiated from the first-order short section drowsiness level B. Thus, the comparison is easy. When the time T4 is longer than 60 minutes, however, the calculation result of the long section drowsiness level D may include a behavior or state of the user different from usual, and the long section drowsiness level D can hardly be regarded as the index value showing the normal drowsiness level of the user. In this case, the accuracy of the result of estimation of the drowsiness level change may decrease. Therefore, the time T4 is preferably 60 minutes at the maximum.

The drowsiness level change estimation unit 103e estimates a change in the drowsiness level of the user. The drowsiness level change estimation unit 103e estimates an increase or decrease in the drowsiness level as the change in the drowsiness level. The drowsiness level change estimation unit 103e estimates the degree of the change in the drowsiness level as the change in the drowsiness level. The drowsiness level change estimation unit 103e can estimate both the increase and the decrease in the drowsiness level. Since the concept of estimation is basically the same, the following description is directed to the estimation of the increase in the drowsiness level.

The drowsiness level change estimation unit 103e estimates whether the drowsiness level increases based on the short section drowsiness level calculated by the short section drowsiness level calculation unit 103g and the long section drowsiness level D calculated by the long section drowsiness level calculation unit 103d. The drowsiness level change estimation unit 103e may use either of the first-order short section drowsiness level B and the second-order short section drowsiness level C as the short section drowsiness level. The description is continued under the assumption that the first-order short section drowsiness level B is used.

The drowsiness level change estimation unit 103e estimates the increase in the drowsiness level using a current first-order short section drowsiness level B at the time of estimation and a past long section drowsiness level D. The current first-order short section drowsiness level B at the time of estimation refers to a real-time (including latest) first-order short section drowsiness level B input from the first-order calculation unit 103b to the drowsiness level change estimation unit 103e at the time of estimation. The drowsiness level change estimation unit 103e uses a long section drowsiness level D in the past time frame as the long section drowsiness level D. Since the long section drowsiness level D is used as a target of comparison with the current drowsiness level, the long section drowsiness level D is preferably the one calculated from measurement data in a time frame in which the drowsiness level does not relatively increase. Thus, the drowsiness level change estimation unit 103e uses a long section drowsiness level D in a time frame in which the drowsiness level is lowest or relatively low in the time-series data of the long section drowsiness levels D that are calculated by the long section drowsiness level calculation unit 103d and older than the current long section drowsiness level D at the time of estimation.

The drowsiness level change estimation unit 103e desirably uses, as the long section drowsiness level D, a long section drowsiness level D calculated using time-series data of past single-point drowsiness levels A that do not include the single-point drowsiness levels A used for calculation of the current first-order short section drowsiness level B at the time of estimation. In the example of FIG. 14, when the drowsiness level change estimation unit 103e estimates the increase in the drowsiness level at the timing at which the first-order short section drowsiness level B(1) is output, the drowsiness level change estimation unit 103e desirably estimates the increase in the drowsiness level using the first-order short section drowsiness level B(1) and a long section drowsiness level Da. The long section drowsiness level Da is calculated using time-series data of single-point drowsiness levels A within a past time T4a that do not include the single-point drowsiness levels A(1), . . . , A(n) used for the calculation of the first-order short section drowsiness level B(1).

If the long section drowsiness level D to be used for the estimation of the increase in the drowsiness level is calculated by including the single-point drowsiness levels A(1), . . . , A(n) used for the calculation of the first-order short section drowsiness level B(1), the calculated long section drowsiness level D is affected by the drowsiness level at the current time point. Therefore, the long section drowsiness level D is desirably calculated using the time-series data of the past single-point drowsiness levels A that do not include the single-point drowsiness levels A(1), . . . , A(n) used for the calculation of the first-order short section drowsiness level B(1). Although the accuracy decreases, the long section drowsiness level D may be calculated by including the single-point drowsiness levels A(1), . . . , A(n).

Although the description has been made about the example in which the long section drowsiness level D is calculated based on the single-point drowsiness levels A, the long section drowsiness level D may be calculated based on the short section drowsiness levels such as the first-order short section drowsiness levels B or the second-order short section drowsiness levels C as described above. When the long section drowsiness level D is calculated based on the short section drowsiness levels, the long section drowsiness level D can be calculated with high accuracy without including the current short section drowsiness level at the time of estimation, but may be calculated by including the current short section drowsiness level at the time of estimation.

Based on the result of observation of the working user, the inventors have considered that, to arouse the user with an increasing drowsiness level, it is optimum to supply an airflow or propose a rest once or twice every 15 to 60 minutes and repeat this operation. Therefore, the long section drowsiness level D to be used for the estimation of the change in the drowsiness level is a past long section drowsiness level D that was calculated during measurement on the same day as the day of the estimation and does not include the current short section drowsiness level at the time of estimation. In one example, when the current short section drowsiness level at the time of estimation is based on measurement data within 1 minute, the long section drowsiness level D is calculated based on measurement data in the past time that does not include the 1 minute, preferably measurement data within, for example, 3 to 30 minutes immediately before the 1 minute.

The drowsiness level change estimation unit 103e repeatedly estimates and outputs the change in the drowsiness level at any appropriate timing. The following value may be used as the long section drowsiness level D to be used for second estimation onward. The drowsiness level change estimation unit 103e may use, as the long section drowsiness level D to be used for the second estimation onward, the long section drowsiness level D used in the previous estimation that the drowsiness level does not increase. In this case, there is an advantageous effect in that the drowsiness level change estimation unit 103e can be prevented from erroneously estimating that the drowsiness level does not increase though the drowsiness level increases in actuality.

A method for estimating the increase in the drowsiness level using the first-order short section drowsiness level B and the long section drowsiness level D is described below. In this estimation, a drowsiness level increase index value is used. This is expressed by a multiple of the first-order short section drowsiness level B relative to the long section drowsiness level D. The drowsiness level increase index value is calculated by “short section drowsiness level/long section drowsiness level.” When the drowsiness level is, for example, expressed in 100 levels from 1 to 100 and the value increases as the drowsiness level increases, the drowsiness level increase index value may range from 0 to 100. The drowsiness level increase index value is 1.0 when the long section drowsiness level D is equal to the first-order short section drowsiness level B and the drowsiness level does not change, and is away from 1.0 as the degree of the increase in the drowsiness level increases. In the actual measurement during normal work or study, the first-order short section drowsiness level B may have a value that is approximately a half to a double of the long section drowsiness level D serving as the reference, and the drowsiness level increase index value may range from 0.5 to 2.0.

When the drowsiness level increase index value is equal to or larger than a threshold, the drowsiness level change estimation unit 103e estimates that the drowsiness level increases. For example, the threshold is optimally a value of 1.1 to 1.7. When the threshold is larger than 1.7, there is an increasing risk that estimation cannot be made that the drowsiness level increases unless the drowsiness level increases considerably. Although the threshold may be increased so that the apparatus control frequency is reduced, the threshold is preferably 1.7 or less for at least the drowsiness level increase estimation.

When the threshold is 1.1 or more, the apparatus including the drowsiness level estimation device 1 can perform apparatus control to arouse the user under the estimation that the drowsiness level increases at a stage at which the drowsiness level does not increase so greatly. Therefore, the current drowsiness level can be maintained. The drowsiness level estimation device 1 uses the method in which the drowsiness level estimation accuracy is increased by indexation of the brain activity level. When the threshold is smaller than 1.1, however, the drowsiness level estimation device 1 may erroneously estimate that the drowsiness level increases at a state at which the drowsiness level does not increase in actuality. Therefore, the threshold is 1.1 at the minimum. When the threshold is a value of 1.2 to 1.5, it is possible to avoid the risk that the result of estimation that the drowsiness level increases can be obtained only when the drowsiness level increases greatly, and to avoid the erroneous estimation.

As described above, the drowsiness level increase index value is 1.0 when the long section drowsiness level D is equal to the first-order short section drowsiness level B, and is larger than 1.0 when the drowsiness level increases. In a specific example, the drowsiness level is expressed in 100 levels from 1% to 100%. For example, when the long section drowsiness level D serving as the reference is 40% and the first-order short section drowsiness level B is 45%, the multiple is 1.125. When the first-order short section drowsiness level B is 70%, the multiple is 1.75. In this case, the threshold for the estimation that the drowsiness level increases is a value close to 1.1 when the drowsiness level increase is to be detected quickly to increase the control frequency. That is, an increase of 5% or more from the reference of 40% is detected and determination is made that the drowsiness level increases.

The threshold for the estimation that the drowsiness level increases is a value close to 1.7 when the detection is to be made after the drowsiness level increases greatly to reduce the control frequency. That is, when the drowsiness level increase index value increases by 30% or more from the reference of 40%, determination is made that the drowsiness level increases. When the drowsiness level is expressed in 10 levels from 1 to 10, the drowsiness level increase index value may have a value of 0 to 10 by calculation. For example, when the long section drowsiness level D serving as the reference is 4 and the first-order short section drowsiness level B is 5, the multiple is 1.25. When the first-order short section drowsiness level B is 7, the multiple is 1.75. Also in this case, the threshold for the estimation that the drowsiness level increases can be set under the concept similar to that for the case of 100 levels.

The drowsiness level change estimation unit 103e outputs “1” when the drowsiness level increase index value is equal to or larger than the threshold and estimation is made that the drowsiness level increases, and outputs “0” when the drowsiness level increase index value is smaller than the threshold and estimation is made that the drowsiness level does not increase. The drowsiness level change estimation unit 103e may output the drowsiness level increase index value as the estimation result. The value output from the drowsiness level change estimation unit 103e is input to the drowsiness level information output unit 103f. When the drowsiness level increase index value is smaller than a threshold smaller than 1, the drowsiness level change estimation unit 103e may estimate that the drowsiness level decreases.

When estimating the increase in the drowsiness level, the drowsiness level change estimation unit 103e uses, as the target of comparison, the long section drowsiness level D calculated based on the measurement data, and does not use a preset value. This is because the normal drowsiness level differs depending on users and accurate estimation cannot be made for a large number of unspecified users if the preset value is used for the estimation of the increase in the drowsiness level. If the increase in the drowsiness level cannot be estimated accurately, the apparatus including the drowsiness level estimation device 1 may excessively perform the apparatus control or may hardly perform the apparatus control. Therefore, the drowsiness level change estimation unit 103e uses the long section drowsiness level D for the estimation of the increase in the drowsiness level. Thus, the drowsiness level change estimation unit 103e can perform appropriate estimation even for unspecified measurement users.

As described above, the drowsiness level information output unit 103f may output any one of the single-point drowsiness level A, the first-order short section drowsiness level B, and the second-order short section drowsiness level C as the drowsiness level information, or may output part or all of them. The drowsiness level information output unit 103f outputs drowsiness level information showing that the drowsiness level increases when “1” is input from the drowsiness level change estimation unit 103e, and outputs drowsiness level information showing that the drowsiness level does not increase when “0” is input from the drowsiness level change estimation unit 103e. As an additional method, the drowsiness level information output unit 103f may set a small threshold to further increase the estimation accuracy, count the number of times “1” is input from the drowsiness level change estimation unit 103e, and determine that the drowsiness level increases when the observed count is plural. Thus, the apparatus including the drowsiness level estimation device 1 suppresses the frequent control change and improves the control accuracy.

When the drowsiness level increase index value is output from the drowsiness level change estimation unit 103e, the drowsiness level information output unit 103f may output the drowsiness level increase index value as the drowsiness level information. As described above, the drowsiness level increase index value is 1 when the drowsiness level does not change, and is away from 1.0 as the degree of the increase in the drowsiness level increases. For example, when the multiple of the first-order short section drowsiness level B relative to the long section drowsiness level D is 2, the increase index value is 2.0. Therefore, the increase index value is larger than 1.0 when the drowsiness level increases. Thus, the drowsiness level information output unit 103f can output the degree of the increase in the drowsiness level as the drowsiness level information using the drowsiness level increase index value.

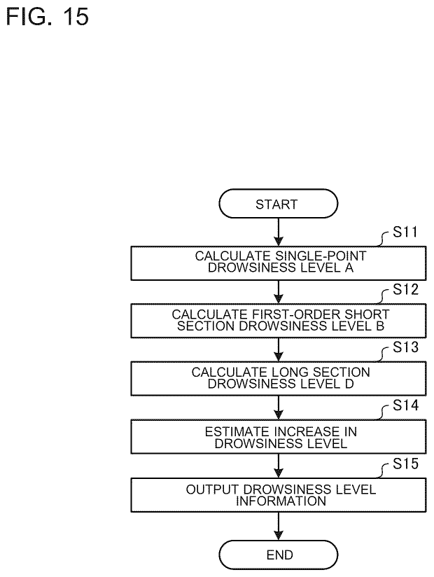

FIG. 15 is a flowchart illustrating an outline of the process in the analysis unit 103 of the drowsiness level estimation device 1 according to Embodiment 3. The analysis unit 103 calculates a single-point drowsiness level A (Step S11). Then, the analysis unit 103 calculates a first-order short section drowsiness level B based on time-series data of the single-point drowsiness levels A calculated in Step S11 (Step S12). The analysis unit 103 calculates a long section drowsiness level D based on time-series data of the single-point drowsiness levels A calculated in Step S11 or the first-order short section drowsiness levels B calculated in Step S12 (Step S13). The analysis unit 103 estimates an increase in the drowsiness level in the manner described above based on the first-order short section drowsiness level B and the long section drowsiness level D (Step S14), and outputs drowsiness level information (Step S15). In the flowchart of FIG. 15, the calculation of the second-order short section drowsiness level C is omitted. When the second-order short section drowsiness level C is used for the estimation of the increase in the drowsiness level, the analysis unit 103 calculates the second-order short section drowsiness level C.

As described above, the analysis unit 103 may output, as the drowsiness level information, part or all of the single-point drowsiness level A, the first-order short section drowsiness level B, and the second-order short section drowsiness level C, or information as to whether the drowsiness level increases. The analysis unit 103 can change the information to be output as appropriate depending on the apparatus including the drowsiness level estimation device 1.

The analysis unit 103 may output drowsiness level information showing whether the drowsiness level is high or low based on any one drowsiness level out of the single-point drowsiness level A, the first-order short section drowsiness level B, and the second-order short section drowsiness level C. For example, the analysis unit 103 may perform the following to estimate whether the drowsiness level is high or low. In a case where the drowsiness level is expressed by a grade value and the value increases as the drowsiness level increases, the analysis unit 103 may estimate that the drowsiness level is high when the estimated drowsiness level is larger than a threshold, and may estimate that the drowsiness level is low when the estimated drowsiness level is smaller than or equal to the threshold.

The analysis unit 103 described above can output the drowsiness level information every 1 second after the measurement of the pulse wave of the user is started by the Doppler sensor 10 and the single-point drowsiness level A is calculated firstly. The analysis unit 103 may set the timing to output the drowsiness level information to a timing after the interval of 1 second or to a set timing.

For example, when repeatedly outputting the drowsiness level information as to whether the drowsiness level increases, the analysis unit 103 may set the time T2 as the set timing and output the drowsiness level information at intervals of the time T2. As described above, the short section drowsiness level is necessary for the estimation as to whether the drowsiness level increases, and the time T2 is a time necessary for the acquisition of the time-series data of the single-point drowsiness levels A necessary for the calculation of the first-order short section drowsiness level B. In other words, the time T2 is the minimum time necessary for the calculation of the first-order short section drowsiness level B. When the analysis unit 103 outputs the drowsiness level information at intervals of the time T2, the analysis unit 103 can output estimation results of the change in the drowsiness level based on the first-order short section drowsiness levels B calculated with all the time-series data of the single-point drowsiness levels A replaced every time. When repeatedly estimating the increase in the drowsiness level, the analysis unit 103 can accurately estimate a decrease in the drowsiness level using the first-order short section drowsiness levels B calculated with all the time-series data of the single-point drowsiness levels A replaced every time.

In the above, the analysis unit 103 can adjust the timing to output the drowsiness level information. However, the drowsiness level information may be output every time the drowsiness level is estimated, and the apparatus that acquires the drowsiness level information may select necessary drowsiness level information and use it for the apparatus control.

As described above, the drowsiness level estimation device 1 of Embodiment 3 can obtain the advantageous effects similar to those of Embodiment 1 or 2, and output not only the single-point drowsiness level A but also the first-order short section drowsiness level B or the second-order short section drowsiness level C as the drowsiness level information. The drowsiness level estimation device 1 can estimate the change in the drowsiness level based on the short section drowsiness level and the long section drowsiness level for each user, and accurately output the drowsiness level information showing the estimation result in real time at an appropriate frequency.

### Embodiment 4

Embodiment 4 relates to an apparatus including any one of the drowsiness level estimation devices 1 of Embodiments 1 to 3. In particular, description is made about a case where the apparatus is an air-conditioning apparatus.

### <Configuration of Air-Conditioning Apparatus 201>

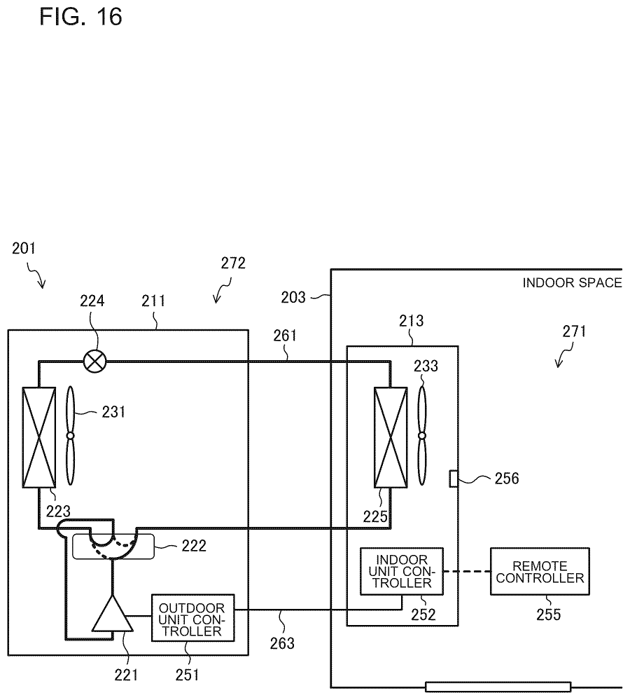

FIG. 16 is a diagram illustrating the configuration of an air-conditioning apparatus 201 according to Embodiment 4. The air-conditioning apparatus 201 is equipment that conditions air in an indoor space 271 that is an air-conditioning target space. Air-conditioning refers to adjustment of, for example, the temperature, humidity, cleanliness, and flow of air in the air-conditioning target space, and is specifically heating, cooling, dehumidifying, humidifying, and air cleaning.

As illustrated in FIG. 16, the air-conditioning apparatus 201 is installed in a building 203. The air-conditioning apparatus 201 is heat-pump air-conditioning equipment that uses, for example, hydrofluorocarbon (HFC) as refrigerant. The air-conditioning apparatus 201 includes a vapor compression refrigerant circuit, and operates by electric power supplied from, for example, a commercial power supply, power generation equipment, or power storage equipment (not illustrated).

As illustrated in FIG. 16, the air-conditioning apparatus 201 includes an outdoor unit 211 provided outside the building 203, an indoor unit 213 provided inside the building 203, and a remote controller 255 to be operated by a user. The outdoor unit 211 and the indoor unit 213 are connected via a refrigerant pipe 261 through which the refrigerant flows and a communication line 263 through which various signals are transferred. The air-conditioning apparatus 201 cools the indoor space 271 by blowing conditioned air such as cold air from the indoor unit 213, and heats the indoor space 271 by blowing hot air.

The outdoor unit 211 includes a compressor 221, a four-way valve 222, an outdoor heat exchanger 223, an expansion valve 224, an outdoor fan 231, and an outdoor unit controller 251. The indoor unit 213 includes an indoor heat exchanger 225, an indoor fan 233, an indoor unit controller 252, and a human detection sensor 256. The refrigerant pipe 261 annularly connects the compressor 221, the four-way valve 222, the outdoor heat exchanger 223, the expansion valve 224, and the indoor heat exchanger 225. The air-conditioning apparatus 201 includes a refrigerant circuit defined by connecting the compressor 221, the four-way valve 222, the outdoor heat exchanger 223, the expansion valve 224, and the indoor heat exchanger 225 by the refrigerant pipe 261. The refrigerant circulates through the refrigerant circuit and operations of a refrigeration cycle are performed.

The compressor 221 compresses the refrigerant and circulates it through the refrigerant pipe 261. Specifically, the compressor 221 compresses low-temperature and low-pressure refrigerant, and discharges high-pressure and high-temperature refrigerant to the four-way valve 222. The compressor 221 includes an inverter circuit that can change an operation capacity depending on a driving frequency. The operation capacity refers to an amount of refrigerant sent out by the compressor 221 per unit time. The compressor 221 changes the operation capacity in response to instructions from the outdoor unit controller 251.

The four-way valve 222 is installed on a discharge side of the compressor 221. The four-way valve 222 changes the refrigerant flow direction in the refrigerant pipe 261 depending on whether the operation of the air-conditioning apparatus 201 is a cooling or dehumidifying operation or a heating operation. The expansion valve 224 is installed between the outdoor heat exchanger 223 and the indoor heat exchanger 225, and expands the refrigerant flowing through the refrigerant pipe 261 by reducing pressure. The expansion valve 224 is an electronic expansion valve that can be controlled so that its opening degree can be changed. The expansion valve 224 adjusts the pressure of the refrigerant by changing the opening degree in response to instructions from the outdoor unit controller 251.

The outdoor heat exchanger 223 exchanges heat between the refrigerant flowing through the refrigerant pipe 261 and air in an outdoor space (external space) 272 outside the indoor space 271. The outdoor fan 231 is provided near the outdoor heat exchanger 223, sucks air in the outdoor space 272, and sends the sucked air to the outdoor heat exchanger 223. The air sent to the outdoor heat exchanger 223 exchanges heat with the refrigerant flowing through the refrigerant pipe 261, and is then blown to the outdoor space 272.

The indoor heat exchanger 225 exchanges heat between the refrigerant flowing through the refrigerant pipe 261 and air in the indoor space 271. The indoor fan 233 is provided near the indoor heat exchanger 225, sucks air in the indoor space 271, and sends the sucked air to the indoor heat exchanger 225. The air sent to the indoor heat exchanger 225 exchanges heat with the refrigerant flowing through the refrigerant pipe 261, and is then blown to the indoor space 271. The air that exchanges heat in the indoor heat exchanger 225 is supplied to the indoor space 271 as conditioned air. Thus, the air in the indoor space 271 is conditioned.

The outdoor unit controller 251 controls the operation of the outdoor unit 211. The indoor unit controller 252 controls the operation of the indoor unit 213.

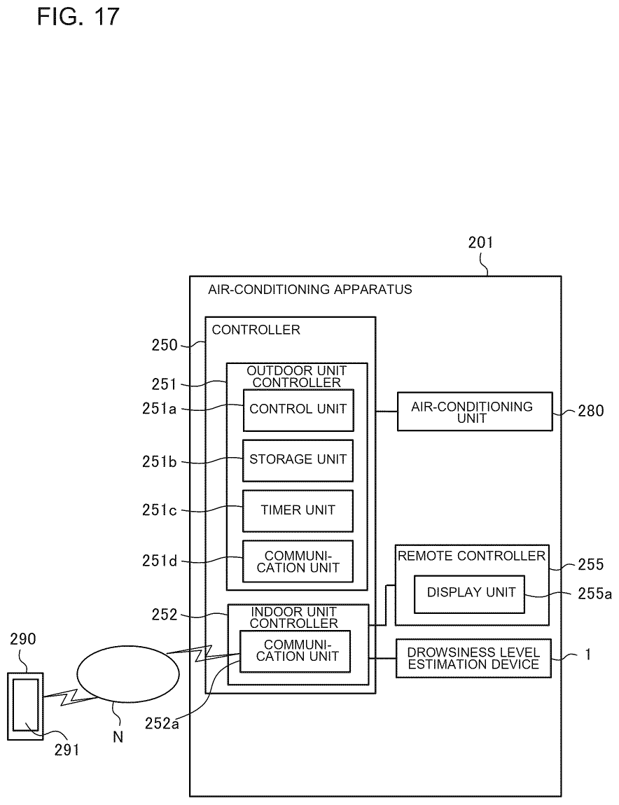

The remote controller 255 is located in the indoor space 271. The remote controller 255 transmits and receives various signals with the indoor unit controller 252 of the indoor unit 213. The remote controller 255 includes a display unit 255a described later as illustrated in FIG. 17. The display unit 255a includes a touchscreen, a liquid crystal display, and light emitting diodes (LEDs). The remote controller 255 has push buttons (not illustrated). The remote controller 255 functions as a command reception unit that receives various commands from the user, and a display unit that displays various types of information for the user. The user operates the remote controller 255 to input commands to the air-conditioning apparatus 201. Examples of the commands include switching commands for operation and stop, and switching commands for operation modes, set temperature, set humidity, air volumes, air directions, and a timer. The air-conditioning apparatus 201 operates in response to the input commands.

FIG. 17 is a block diagram of the air-conditioning apparatus 201 according to Embodiment 4. The air-conditioning apparatus 201 includes a controller 250, an air-conditioning unit 280, and any one of the drowsiness level estimation devices 1 of Embodiments 1 to 3. An information apparatus 290 to be operated by the user is connected to the air-conditioning apparatus 201 via a network N.

The controller 250 controls the overall air-conditioning apparatus 201. The controller 250 controls the operation of an apparatus body, in other words, the air-conditioning unit 280 based on drowsiness level information output from the drowsiness level estimation device 1. The controller 250 includes the outdoor unit controller 251 and the indoor unit controller 252 described above. Although illustration is omitted in FIG. 17, the controller 250 includes the control detail determination unit 104 and the apparatus control unit 105 described in Embodiment 1. The control detail determination unit 104 and the apparatus control unit 105 may be provided to either of the outdoor unit controller 251 and the indoor unit controller 252.

The outdoor unit controller 251 includes a control unit 251a, a storage unit 251b, a timer unit 251c, and a communication unit 251d. Those units are connected via a bus (not illustrated).

The control unit 251a controls the overall outdoor unit. The storage unit 251b is a memory such as a RAM or a ROM, and stores data necessary for control. The timer unit 251c measures time. The communication unit 251d is an interface for communication with the indoor unit controller 252 via the communication line 263 (see FIG. 16).

As illustrated in FIG. 16, the outdoor unit controller 251 is connected to the indoor unit controller 252 by the communication line 263. The outdoor unit controller 251 cooperates with the indoor unit controller 252 by receiving various signals from the indoor unit controller 252 via the communication line 263.

The indoor unit controller 252 includes a communication unit 252a that communicates with the outdoor unit controller 251 and the remote controller 255. The communication unit 252a is an interface for communication with the outdoor unit controller 251 and the remote controller 255. The communication unit 252a is further connected to the information apparatus 290 via the network N. The communication unit 252a performs a process of receiving various user commands from the remote controller 255, and a process of transmitting the various commands received from the remote controller 255 to the indoor unit controller 252. The communication unit 252a performs a process of transmitting notification information for the user to the remote controller 255.

The outdoor unit controller 251 and the indoor unit controller 252 are microprocessor units. Each of the outdoor unit controller 251 and the indoor unit controller 252 includes a central processing unit (CPU), a read only memory (ROM), and a random access memory (RAM). The ROM stores a control program or other data. The outdoor unit controller 251 and the indoor unit controller 252 are not limited to the microprocessor units. For example, each of the outdoor unit controller 251 and the indoor unit controller 252 may be firmware that can be updated. Each of the outdoor unit controller 251 and the indoor unit controller 252 may be a program module to be executed by a command from a CPU (not illustrated) or other devices. Although the description has been made about the example in which the controller 250 includes the outdoor unit controller 251 and the indoor unit controller 252 that are separately provided in the outdoor unit 211 and the indoor unit 213, respectively, the outdoor unit controller 251 and the indoor unit controller 252 may be provided as a single control unit having their functions.

The air-conditioning unit 280 conditions air in the indoor space 271, and corresponds to the refrigerant circuit, the outdoor fan 231, and the indoor fan 233 in FIG. 16.

The air-conditioning apparatus 201 configured as described above controls the operation of the air-conditioning unit 280 based on drowsiness level information output from the drowsiness level estimation device 1. Specifically, when the drowsiness level information shows that the drowsiness level is high, the controller 250 of the air-conditioning apparatus 201 controls the air-conditioning unit 280 to perform an operation of arousing the user. Hitherto, it has been ascertained that the user is aroused by wind. Therefore, when the drowsiness level of the user is high, the controller 250 increases the air sending amount by, for example, increasing the rotation speed of the indoor fan 233 as an arousing operation for arousing the user.

The air-conditioning apparatus 201 includes the human detection sensor 256 that detects the position of the user. When the drowsiness level of the user is high, the controller 250 controls the air-conditioning unit 280 based on the position of the user detected by the human detection sensor 256 to perform the following arousing operation for arousing the user. The controller 250 controls a swing operation for vertically moving a vertical airflow direction flap (not illustrated) provided to the indoor unit 213 so that the user is intermittently exposed to airflow, or controls a swing operation for laterally moving a lateral airflow direction flap (not illustrated) so that the user is intermittently exposed to airflow. Through the above control, the air-conditioning apparatus 201 can arouse the user with the high drowsiness level.

The room temperature is preferably lower by about 1 degree Celsius than the temperature that the user feels as an optimum temperature because of an effect that the brain is cooled. Thus, the work efficiency is high. Particularly during heating, the drowsiness level increases when the set temperature is excessively high. Therefore, the controller 250 may perform, as the arousing operation, control for adjusting the set indoor temperature to a temperature slightly lower than the current temperature.

When the drowsiness level is conversely low, the controller 250 reduces the air sending amount by reducing the rotation speed of the indoor fan 233, or controls the vertical airflow direction flap to keep an upward posture to the extent possible so that the user is not exposed to airflow. The controller 250 controls either or both of the vertical airflow direction flap (not illustrated) and the lateral airflow direction flap (not illustrated) so that the user is not exposed to airflow. Through the above control, the air-conditioning apparatus 201 can reduce the case where the concentration level decreases because the user exposed to airflow turns attention to the airflow. In other words, the air-conditioning apparatus 201 can obtain an advantageous effect in that the user with the low drowsiness level and the high concentration level can work without paying attention to the airflow.

When the drowsiness level estimation device 1 is the drowsiness level estimation device 1 of Embodiment 3, the drowsiness level shown by the drowsiness level information output from the drowsiness level estimation device 1 includes the single-point drowsiness level A, the first-order short section drowsiness level B, and the second-order short section drowsiness level C. If the air-conditioning apparatus 201 controls the operation of the air-conditioning unit 280 based on the single-point drowsiness level A, that is, the drowsiness level of the user at a certain time point, the control of the air-conditioning apparatus 201 may be changed frequently. In this case, the user may be annoyed with the operation of the air-conditioning apparatus 201 or dissatisfied frequently with the change in temperature or airflow. In view of this, the air-conditioning apparatus 201 uses the first-order short section drowsiness level B or the second-order short section drowsiness level C to perform control based on the drowsiness level of the user. Thus, there is an advantage in that the control can be determined at an appropriate frequency with the tendency reflected accurately.

However, the second-order short section drowsiness level C is a value calculated by performing the averaging process twice. Therefore, when the air-conditioning apparatus 201 performs control based on the second-order short section drowsiness level C, it is difficult to perform control with a quick response to fine drowsiness level increases compared with the control based on the first-order short section drowsiness level B. Since the second-order short section drowsiness level C is a value calculated by performing the averaging process twice, the calculation time is required compared with the first-order short section drowsiness level B. Therefore, when the second-order short section drowsiness level C is used as the drowsiness level of the user, the air-conditioning apparatus 201 requires time for the control change estimation. Thus, the air-conditioning apparatus 201 preferably selects the first-order short section drowsiness level B or the second-order short section drowsiness level C depending on a desired control change frequency, response, and estimation time.

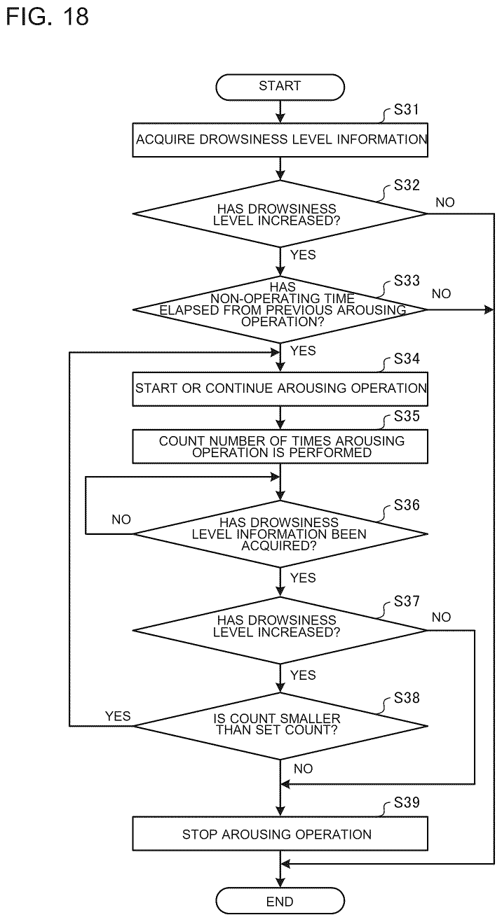

The air-conditioning apparatus 201 can also control the operation of the air-conditioning unit 280 to perform the arousing operation in response to the change that is the increase in the drowsiness level. The control in this case is described with reference to FIG. 18.

FIG. 18 is a flowchart illustrating the operation of the air-conditioning apparatus 201 according to Embodiment 4. In the following description, the time T2 is the same as the time T2 in Embodiment 3. The air-conditioning apparatus 201 acquires drowsiness level information output from the drowsiness level estimation device 1 (Step S31). The air-conditioning apparatus 201 acquires drowsiness level information output at intervals of the time T2 from the drowsiness level estimation device 1. In the following description, drowsiness level information R1, drowsiness level information R2, drowsiness level information R3, and drowsiness level information R4 are first, second, third, and fourth pieces of drowsiness level information output at intervals of the time T2 from the drowsiness level estimation device 1 in this order.

The air-conditioning apparatus 201 determines whether the drowsiness level information R1 acquired in Step S31 shows that the drowsiness level of the user increases (Step S32). When the drowsiness level information R1 shows that the drowsiness level increases, the air-conditioning apparatus 201 controls, through Step S33 described later, the air-conditioning unit 280 to start an arousing operation for arousing the user (Step S34). As described above, the arousing operation is the swing operation for vertically moving the vertical airflow direction flap. Then, the air-conditioning apparatus 201 counts the number of times the arousing operation is performed (Step S35). In this case, the air-conditioning apparatus 201 counts “1.”

The air-conditioning apparatus 201 continues the arousing operation until the next drowsiness level information R2 is acquired. When the drowsiness level information R2 is newly acquired (Step S36), the air-conditioning apparatus 201 determines whether the drowsiness level information R2 shows that the drowsiness level of the user increases (Step S37). When the drowsiness level information R2 shows that the drowsiness level still increases, the air-conditioning apparatus 201 determines whether the count is smaller than a set count (Step S38). If the set count is “3,” the current count is “1” and is smaller than the set count. Therefore, the air-conditioning apparatus 201 returns to Step S34 and continues the arousing operation. The air-conditioning apparatus 201 may continue the arousing operation at the same air volume as that when the arousing operation was started previously in Step S34, or may increase the air volume to arouse the user more intensely. The air-conditioning apparatus 201 preferably increases the air volume because the drowsiness level of the user can be reduced quickly. Then, the air-conditioning apparatus 201 counts “2” as the number of times the arousing operation is performed (Step S35).

The air-conditioning apparatus 201 continues the arousing operation until the next drowsiness level information R3 is acquired. When the drowsiness level information R3 is newly acquired (Step S36), the air-conditioning apparatus 201 determines whether the drowsiness level information R3 shows that the drowsiness level of the user increases (Step S37). When the drowsiness level information R3 shows that the drowsiness level still increases, the air-conditioning apparatus 201 determines whether the count is smaller than the set count (Step S38). The current count is “2” and is smaller than the set count “3.” Therefore, the air-conditioning apparatus 201 returns to Step S34 and continues the arousing operation. The air-conditioning apparatus 201 may continue the arousing operation at the same air volume as that when the arousing operation was continued previously in Step S34, or may increase the air volume to arouse the user more intensely. Then, the air-conditioning apparatus 201 counts “3” as the number of times the arousing operation is performed (Step S35).

The air-conditioning apparatus 201 continues the arousing operation until the next drowsiness level information R4 is acquired. When the drowsiness level information R4 is newly acquired (Step S36), the air-conditioning apparatus 201 determines whether the drowsiness level information R4 shows that the drowsiness level of the user increases (Step S37). When the drowsiness level information R4 shows that the drowsiness level still increases, the air-conditioning apparatus 201 determines whether the count is smaller than the set count (Step S38). The count is “3” and is not smaller than the set count. Therefore, the air-conditioning apparatus 201 stops the arousing operation (Step S39).

That is, when the drowsiness level of the user does not decrease though the set count of arousing operations is performed, the air-conditioning apparatus 201 stops the arousing operation because the user may be dissatisfied with the feeling of airflow. Although the set count is “3,” the set count is not limited to “3.” The air-conditioning apparatus 201 performs the swing operation as the arousing operation, but may perform control for reducing the room temperature to cool the brain though the control requires time to arouse the user. That is, the above arousing operation includes the operation of reducing the room temperature as well as the swing operation.

When the drowsiness level information R2 or R3 does not show that the drowsiness level of the user increases, that is, the user is aroused in Step S37, the air-conditioning apparatus 201 stops the arousing operation without returning to the arousing operation in Step S34 (Step S39).

Step S33 is described. When the air-conditioning apparatus 201 estimates in Step S32 that the drowsiness level increases, the air-conditioning apparatus 201 does not immediately perform the arousing operation, but performs the arousing operation when determination is made that a non-operating time has elapsed from the previous arousing operation. That is, the air-conditioning apparatus 201 sets the non-operating time so that the arousing operation is not performed for a predetermined time after the arousing operation has once been performed in response to the estimation that the drowsiness level increases. This is because the user is kept aroused after he/she has been aroused by the arousing operation of the air-conditioning apparatus 201.

If the non-operating time is not provided, the air-conditioning apparatus 201 may frequently expose the user to airflow, and the comfort may be impaired. With the non-operating time, the air-conditioning apparatus 201 can avoid the user's dissatisfaction with airflow or thermal sensation. For example, the non-operating time is preferably 10 minutes to 15 minutes because a decrease in the drowsiness level is observed clearly.

As described above, the air-conditioning apparatus 201 can accurately perform the arousing operation at an appropriate frequency depending on the drowsiness level of the user by controlling the air-conditioning unit 280 using the drowsiness level information output from the drowsiness level estimation device 1. As a result, the air-conditioning apparatus 201 can maintain the work efficiency of the user during work or study. Alternatively, the air-conditioning apparatus 201 has an advantageous effect in that the work efficiency can be improved by suppressing the increase in the drowsiness level of the user.

The remote controller 255 is part of the components of the air-conditioning apparatus 201, and the information apparatus 290 is an apparatus owned by the user. The information apparatus 290 includes a display unit 291 such as a liquid crystal panel. The display unit 291 displays various types of information. The information apparatus 290 is, for example, a smartphone or a tablet. An application for displaying the drowsiness level of the user or other information is installed in the information apparatus 290. An air-conditioning control application may be installed and the information apparatus 290 may be used in place of the remote controller 255.

When the information apparatus 290 is operated by the user, the information apparatus 290 starts the application, acquires drowsiness level information output from the drowsiness level estimation device 1 via the network N, and displays it on the display unit 291. As specific control, the controller 250 of the air-conditioning apparatus 201 performs a process of transmitting, to the information apparatus 290 via the communication unit 252a, drowsiness level information output by the drowsiness level estimation device 1 in response to a request from the information apparatus 290, and displaying the drowsiness level information on the information apparatus 290. Although the drowsiness level information is displayed on the information apparatus 290, the drowsiness level information may be displayed on the display unit 255a of the remote controller 255.

As described above, the air-conditioning apparatus 201 visualizes the drowsiness level information by displaying it on the information apparatus 290 or the display unit 255a of the remote controller 255. Thus, the user can view the drowsiness level.

Although the description has been made that the apparatus including the drowsiness level estimation device 1 is the air-conditioning apparatus 201, the apparatus is not limited to the air-conditioning apparatus 201, and the drowsiness level estimation device 1 can be provided to an electrical apparatus, a vehicle, an amusement apparatus, or various other apparatuses. The drowsiness level estimation device 1 can also be provided to, for example, a labor management apparatus or a learning management apparatus.

The drowsiness level estimation device 1 can be provided to an apparatus in a wide variety of fields such as healthcare, labor, education, sleep, mindfulness, meditation, customer services, marketing, or sport mental training. When the drowsiness level estimation device 1 is provided to such an apparatus, the apparatus can perform the apparatus control using the drowsiness level estimation result and present the drowsiness level estimation result to the user. When the drowsiness level estimation device 1 is applied to various apparatuses, the drowsiness level can be visualized and presented to the user of the apparatus.

1. A drowsiness level estimation device comprising:
a Doppler sensor configured to detect a pulse wave of a user; and
an analysis unit configured to analyze the pulse wave detected by the Doppler sensor, wherein
the analysis unit is configured to generate an index value by digitizing the pulse wave based on chaos analysis in which a source of analysis is a pulse waveform shift that is a time-series shift of a waveform of the pulse wave, estimate a drowsiness level of the user based on the index value, and output drowsiness level information related to the estimated drowsiness level,
wherein the chaos analysis is a process of sequentially performing:
a step of calculating vectors determined from time-series data of the pulse waveform shift and a preset delay time;
a step of generating an attractor having the vectors arranged in time series in a multidimensional state space having three or more dimensions; and
a step of calculating, as a Lyapunov exponent that is the index value, an enlargement factor obtained by giving a hypersphere as an initial state to trajectories of the attractor and repeating an operation of stretching the hypersphere at intervals of a preset slide time based on the trajectories so that the hypersphere turns into an ellipse
the index value shows a degree of separation of the trajectories compared with the initial state, and shows that a brain activity level increases as the index value increases,
the analysis unit is configured to output the drowsiness level information showing that the drowsiness level decreases as the index value increases, and that the drowsiness level increases as the index value decreases, and
the drowsiness level information is information showing a current drowsiness level, time-series drowsiness levels, or both of the current drowsiness level and the time-series drowsiness levels.

2. (canceled)

3. (canceled)

4. The drowsiness level estimation device of claim 1,
wherein
the Doppler sensor is configured to detect a pulse of the user, and
the analysis unit is configured to estimate a brain activity level of the user based on the index value, estimate an autonomic activity level of the user based on the pulse, and estimate the drowsiness level based on the brain activity level and the autonomic activity level.

5. The drowsiness level estimation device of claim 4, wherein
the analysis unit has a first contribution factor showing how greatly the brain activity level affects the drowsiness level, and a second contribution factor showing how greatly the autonomic activity level affects the drowsiness level, the second contribution factor being smaller than the first contribution factor, and
the analysis unit is configured to estimate the drowsiness level based on the first contribution factor and the second contribution factor in addition to the brain activity level and the autonomic activity level.

6. The drowsiness level estimation device of claim 5, wherein
the first contribution factor is 60% or more and 80% or less, and
the second contribution factor is 20% or more and 40% or less.

7. The drowsiness level estimation device of claim 1,
wherein the analysis unit comprises:
a single-point drowsiness level calculation unit configured to calculate a single-point drowsiness level that is a drowsiness level of the user at a certain time point based on the index value;
a short section drowsiness level calculation unit configured to calculate a short section drowsiness level by performing an averaging process on time-series data of the single-point drowsiness levels in a short section;
a long section drowsiness level calculation unit configured to calculate a long section drowsiness level by performing an averaging process on time-series data of the single-point drowsiness levels or the short section drowsiness levels in a long section that is longer than the short section; and
a drowsiness level change estimation unit configured to estimate a change in the drowsiness level of the user based on the short section drowsiness level and the long section drowsiness level.

8. An apparatus comprising:
the drowsiness level estimation device of claim 1, and
a controller configured to control an operation of an apparatus body based on the drowsiness level information output from the drowsiness level estimation device.

9. The apparatus of claim 8, further comprising a display unit configured to perform displaying, wherein
the display unit is configured to display the drowsiness level information output from the analysis unit.

10. An air-conditioning apparatus comprising:
the drowsiness level estimation device claim 1;
an air-conditioning unit configured to condition air in an indoor space; and
a controller configured to control the air-conditioning unit based on the drowsiness level information output from the drowsiness level estimation device.

11. A drowsiness level estimation device comprising:
a Doppler sensor configured to detect a pulse wave of a user; and
an analysis unit configured to analyze the pulse wave detected by the Doppler sensor, wherein
the analysis unit is configured to generate an index value by digitizing the pulse wave based on chaos analysis in which a source of analysis is a pulse waveform shift that is a time-series shift of a waveform of the pulse wave, estimate a drowsiness level of the user based on the index value, and output drowsiness level information related to the estimated drowsiness level,
the Doppler sensor is configured to detect a pulse of the user, and
the analysis unit is configured to estimate a brain activity level of the user based on the index value, estimate an autonomic activity level of the user based on the pulse, and estimate the drowsiness level based on the brain activity level and the autonomic activity level.
